(19) United States
(12) Patent Application Publication
(10) Pub. No.: US 2005/0233955 A1
(43) Pub. Date: Oct. 20, 2005

(54) Stimulation of nerve cell regeneration

(75) Inventors: Jeremy Simon Hudson Taylor, Oxford (GB); Edward Thomas William Bampton, Cambridge (GB)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Isis Innovation Limited, Summertown (GB)

(21) Appl. No.: 10/510,065

(22) PCT Filed: Apr. 4, 2003
(86) PCT No.: PCT/GB03/01498

§ 371 Date: Jun. 6, 2005

(30) Foreign Application Priority Data
Apr. 4, 2002 (GB) 0207829.3

(51) Int. Cl. C12N 5/08; A61K 38/17

(52) U.S. Cl. 514/12; 435/368

(57) ABSTRACT

The invention provides a method for stimulating the regeneration of a neuronal cell, said method comprising exposing the cell to the osteonectin protein or a functional equivalent thereof in an amount that is effective to stimulate the regeneration of the cell.

The invention relates to a method for the stimulation of nerve cell regeneration. More particularly, the invention relates to the use of the osteonectin protein for the stimulation of nerve cell regeneration.

Unlike lower vertebrates, from fish and amphibia to lizards, mammals have through the course of evolution lost the ability to repair damage to the brain and spinal cord. This loss of ability is well illustrated by injury to the visual system across a range of organisms. Optic nerve damage to the goldfish is readily repaired—the axons of the retinal projection neuron, the retinal ganglion cell (RGC), will sprout and send their projections back across the site of the lesion, reform their synaptic connections with their target, the tectum (or superior colliculus) and refine these connections into the appropriate retinotopographic map necessary for functional vision (Bernhardt, 1999). This is an ability shared by amphibia (Gaze, 1970) and the lizard (Stirling et al., 1999). However, once in the mammalian system all that one can observe are the ‘small and frustrated regenerative efforts’of the axons, described by Cajal (Cajal, 1928).

The potential for repair in the central nervous system (CNS) has been demonstrated using grafts derived from the peripheral nervous system (PNS). Unlike the CNS, the PNS is capable of impressive feats of repair (Langley, 1895; Fawcett and Keynes, 1990) and it was thus postulated that transplanted PNS tissue may be able to encourage a more robust regenerative response from central neurons. However, it was not until 1980 that Aguayo and colleagues began systematically to use PNS grafts to examine the regenerative ability of the CNS (Richardson et al., 1980), opening up new vistas of research with the realisation that CNS damage was not as intractable as had been thought.

It appears that it is not so much an intrinsic inability of the CNS neuron to regenerate, but the environment of the CNS which is hostile to regeneration. The problems that the regenerating axon faces in the CNS are summarised by Schwab et al., 1993. Firstly there is axotomy-induced cell death of the neurons themselves. Secondly the gliotic scar that forms at the site of injury provides both a physical and chemical barrier to those axons that do regenerate. Thirdly, the CNS environment itself appears to be inhibitory to growth, or at the very least not to support regeneration.

Repair of the damaged CNS relies on promoting the survival of damaged neurons and on overcoming the intrinsic barriers presented by the hostile nature of the injured CNS. Given the range of barriers the regenerating neuron can encounter, there is a wide range of strategies that have been employed. These include the inhibition of apoptosis, strategies designed to block the effects of growth inhibitory molecules, stem cell therapy (particularly the use of the embryonic stem cell), PNS grafting (including the use of purified Schwann cells), the transplantation of olfactory ensheathing cells (OECs), and neurotrophic factor delivery. However, although the possibility of CNS regeneration has been demonstrated using certain of these methods, these approaches yield only a limited response, with generally only small percentages of fibres regenerating any distance (Vidal-Sanz et al., 1987) and giving limited, if any, functional recovery (e.g. Cheng et al., 1996; Kierstead et al., 1985, 1989; Thanos, 1992).

It is thus clear that although there is a great potential for repair of traumatic CNS damage, there remains a great need for an effective method that allows not only anatomical reconnection, but also functional recovery of CNS function.

It is an object of the invention to provide a method of stimulating the regeneration of neuronal cells. It is another object of the invention to provide a method of stimulating the regeneration of neurons after damage due to physical injury or after damage due to disease. These and other objects of the invention are provided by the embodiments of the invention described below.

The Invention

According to the invention, there is provided a method for stimulating the regeneration of a neuronal cell, said method comprising exposing the cell to the osteonectin protein or a functional equivalent thereof in an amount that is effective to stimulate the regeneration of the cell.

It has been discovered that osteonectin has a dramatic effect on neuronal cells in terms of stimulating their survival and regeneration. This effect is potent, in that small amounts of the protein are neurotrophic, meaning that the observed effect has a direct physiological relevance.

CNS axonal regeneration after injury appears to be inhibited by the negative environment surrounding the injury site. It is considered that this environment may be due to inhibitory proteins present on the surface of oligodendroglial cells and other inhibitory factors present in the nervous system. In spite of this, the action of osteonectin both supports and sustains the process of axonal advance and regeneration.

The fact that CNS axons have an intrinsic capability to regenerate together with the actions of osteonectin documented herein, as well as the feasibility of its delivery, make this protein, and agents based either on this protein or on its upstream or downstream binding partners, ideal tools for reparative treatments by controllably and safely modifying the local environment to the benefit of regenerating fibres, in a region-specific and chemospecific manner.

This effect of osteonectin was noted in pursuing the identity of the factor that is responsible for the biological effects of primary Schwann cell conditioned medium on neurons. Schwann cell medium has previously been known to stimulate the growth of neuronal cells. Biochemical characterisation and comparison of Schwann cell conditioned media was performed by protein electrophoresis. In addition, a radiolabelling study was employed to identify the most likely candidates for the observed biological effects. Enough protein was obtained to sequence a candidate protein. Surprisingly, the component responsible for neuronal cell regeneration was identified by sequence analysis as osteonectin (also known as secreted protein acidic and rich in cysteine (SPARC)).

Osteonectin was first identified in bone as a collagen and hydroxyapatite binding protein, acting as a link between these skeletal components (Termine et al., 1981). The sequence of the human osteonectin protein is given as Genbank accession number XM_{—}032759.1 (nucleotide accession number GI:14722769), whilst the mouse and rat proteins have accession numbers NM 009262 and D28875 respectively. Initial immunohistochemical localisation showed restricted reactivity to bone and dentine in the tooth, but subsequently it has been shown to have a much more widespread expression during development to non-mineralised tissues such as kidney, testis, lung and skin (Mundlos et al., 1992). Although levels of osteonectin are generally much lower in the adult, it is upregulated following injury, where tissue remodelling is occurring, most notably in endothelial cells (Sage et al., 1989). Expression is highest where extracellular matrix (ECM) components are being laid down, both in development and following injury (Brekken and Sage; 2001). Expression and secretion of this protein by Schwann cells has not been reported prior to this study.

Osteonectin has been localised in the CNS previously. It is widely expressed in the developing and adult mouse brain (Mendis and Brown, 1994), in the adult brain showing higher levels in caudal regions and especially localised to astrocytes in synapse rich areas (Mendis et al., 1995). It has also been found in retinal ganglion cells (RGCs) and astrocytes in the bovine retina (Yan et al., 1998) and in the rat retina in the Müller glia and RGCs (Gilbert et al., 1999). Thus it is likely to play a role during nervous system development and possibly in synaptic remodelling, although this has not been explored in detail. An osteonectin knockout mouse has been generated, but so far only examination of bone-related defects has been carried out in detail (Delany et al., 2000), although cataractogenesis, accelerated dermal wound healing and increased fat deposition have also been briefly noted (Bradshaw and Sage, 2001).

Osteonectin itself is a glycoprotein, the core domain being some 32 kDa in mass, but with its sugar moieties attached running on SDS-PAGE at around 4 kDa, hence its alternative name, BM-40 (basement membrane 40). It comprises an acidic N-terminus, a follistatin domain and an E-C domain, each domain mediating a range of the effects of this protein.

Two functions of osteonectin are of particular interest in the context of the discovery that the protein is capable of stimulating the regeneration of neuronal cells. Firstly it is known to bind to and inhibit the activity of certain growth factors, namely platelet derived growth factor-AB and -BB (PDGF-AB, -BB; Raines et al., 1992) and vascular endothelial growth factor (VEGF; Kupprion et al., 1998). Both these factors typically induce cell proliferation, not necessarily a useful trait in repairing terminally differentiated neurons. It can also inhibit the action of FGF-2, though here the mechanism appears to be indirect as there is no evidence for a direct interaction of osteonectin and FGF-2 (Hasselaar and Sage, 1992).

Osteonectin can induce the expression of transforming growth factor-b (TGF-b; Francki et al., 1999). Certainly TGF-b can have neurotrophic actions both on dorsal root ganglion neuron in synergy with NGF (Chalazonitis et al., 1992) and on hippocampal neurons (Abe et al., 1996). The protein may well be acting to effect neuronal survival and regeneration by this mechanism, although the Applicant does not wish to be bound by this theory.

Secondly, osteonectin is well-characterised as a counter-adhesion molecule, thereby modulating cell morphology, migration and differentiation (Bradshaw and Sage, 2001). This anti-adhesive property may help promote axonal extension, preventing over-tight adherence until the target is reached. One can also reconcile this to the need for Schwann cell migration following injury.

As discussed above, in order that neuronal cell regeneration be stimulated, the cell must be exposed to the osteonectin protein or functional equivalent thereof, in an amount that is effective to stimulate the regeneration of the cell. By stimulation of neuronal cell regeneration is meant the enhancement of the survival of neuronal cells and their ability to grow, such that axonal regeneration is promoted. In the context of an injured or diseased nerve, reestablishment of contact with the CNS target may be encouraged. Such an effect can be assayed by the addition of the osteonectin protein or functional equivalent to cell culture medium in which neuronal cells are growing, followed by the monitoring of their regeneration over the course of time. Such an effect can be compared to control cells whose culture medium does not contain the osteonectin protein or functional equivalent.

A particularly surprising element of the present invention is the discovery that osteonectin is able to stimulate the regeneration of CNS neuronal cells, since these cells are normally prevented from regenerating once damaged or diseased. However, it is considered that the osteonectin protein, and functionally equivalent fragments and variants as described herein will also be of considerable utility in the stimulation of regeneration of the cells of the PNS. Accordingly, both CNS and peripheral neuronal cell types are suitable target cells for treatment according to the present invention. Preferably, however, the invention is used for stimulation of the regeneration of CNS neuronal cells.

By “functional equivalent” is meant any compound that is effective to elicit a similar effect to that noted for osteonectin, by the same mechanism of action. For example, functional equivalents according to the invention include fragments and variants of the osteonectin protein, as well as compounds that mimic the structure of the osteonectin protein, or of active fragments of the protein. Such functional equivalents may be polypeptides, but may alternatively take the form of natural or modified substrates, enzymes, receptors, small organic molecules (such as small natural or synthetic organic molecules of up to 2000 Da, preferably 800 Da or less), peptides (such as cyclic peptides), peptidomimetics, antibodies, and other structural or functional mimetics of the osteonectin protein.

By “fragment” is meant any truncated version of the osteonectin protein, either truncated at its C terminus, its N terminus or at both termini, provided that the fragment includes the part of the protein that is responsible for the observed activity in stimulating the regeneration of neuronal cells. Such fragments may be used to effect an equivalent, or even improved biological phenotype as that exhibited by the wild type protein. For example, truncated fragments of the osteonectin protein may exhibit similar properties as the untruncated form, but may possess other improved properties, such as a better pharmacokinetic profile, a longer half-life in the circulation, lower immunogenicity and so on.

The design of such functionally active fragments is perfectly within the ability of the skilled addressee and will involve the rational or random truncation of the full length protein, followed by functional assay. Such truncation techniques are described in detail in widely available text books such as Sambrook et al., Molecular Cloning; A Laboratory Manual, Second Edition (2001), Cold Spring Harbor Laboratory Press.

Variants of the osteonectin protein that include the active site of the protein may also be used to mimic or effect an equivalent, or even improved biological phenotype as the wild type protein. Such variants may be proteins or peptides that are homologous to the osteonectin protein. Two proteins or peptides are said to be “homologous”, as the term is used herein, if the sequence of one of the polypeptides has a high enough degree of identity or similarity to the sequence of the other polypeptide to allow an inference of shared function to be made. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A.M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

As defined herein, variants therefore include natural biological variants (for example, allelic variants or geographical variations within the species from which the polypeptides are derived) and mutants (such as mutants containing amino acid substitutions, insertions or deletions) of the osteonectin protein. Such mutants may include proteins in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

Typically, greater than 80% identity between two proteins or peptides (preferably, over a specified region such as the active site region) is considered to be an indication of functional equivalence. Preferably, functionally equivalent variants according to the invention therefore have a degree of sequence identity with the osteonectin protein, or with active fragments thereof, of greater than 80%. More preferred polypeptides have degrees of identity of greater than 85%, 90%, 95%, 98% or 99%, respectively with the osteonectin protein, or with active fragments thereof. Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

There are numerous situations in which the regeneration of neuronal cells can be stimulated. In order to mediate the stimulation of neuronal cell regeneration, the relevant cells must of course be exposed to the osteonectin protein or functional equivalent thereof in the environment of their surrounding medium.

For example, in one embodiment, neuronal cells may be exposed to osteonectin or a functional equivalent in vitro so as to stimulate their regeneration in tissue culture. The resulting cell mass may then be used for whatever purpose is desired, be this further study and research or transplant into a patient.

It is considered that osteonectin, and the other agents discussed herein are suitable agents for both the therapy and prophylaxis of diseases or injuries where nerve damage occurs. In a therapeutic context, such situations include, but are not limited to diseases including peripheral nerve damage, such as by physical injury or disease state such as diabetes, in the case of injury or a disease state of the CNS, including physical damage to the spinal cord, brain trauma, stroke, retinal and optic nerve lesions, neurodegenerative diseases such as Alzheimer's disease and Parkinson's disease, neuromuscular diseases, autoimmune diseases of the nervous system, tumours of the central nervous system, damage to motor neurons such as occurs in conditions such as amyotrophic lateral sclerosis, and degenerative diseases of the retina such as retinitis pigmentosa and age-related macular degeneration. In these scenarios, it may be preferable to expose neuronal cells to osteonectin or a functional equivalent thereof in situ to stimulate regeneration of the cells such that axonal regeneration is promoted and reestablishment of contact with the CNS target is encouraged.

In therapeutic use, osteonectin, functional equivalents and associated therapeutic drugs can be administered by any route whereby drugs may be conventionally administered. For a brief review of general methods of drug delivery, see for example Langer, Science 249: 527-1533 (1990). Oral administration may lead to degradation in the gastrointestinal canal, and, accordingly, methods relying on this mode of administration must be especially adapted to avoid enzyme-catalyzed degradation and to maintain effective concentrations.

Preferably, the osteonectin protein, or functional equivalent thereof is used therapeutically in conjunction with an artificial bridging substrate such as Elvax®, an Ethylene-vinyl acetate co-polymer (see Bloch et al., 2001, Exp Neurol., 172(2):425; Aebischer et al., 1989, J Neurosci Res. 23(3):282; Fine et al., (2000) Nerve regeneration: Ed. Academic Press. Second edition. Principles of tissue engineering, pp.785-797). Artificial substrates of this nature provide a mechanism for slow release of proteins allowing for a sustained delivery over the period of repair. They also allow cocktails of known beneficial neurotrophins to be delivered simultaneously with the osteonectin protein, or functional equivalent.

In one embodiment of the invention, the osteonectin protein or functional equivalent (such as an active peptide that induces neuronal survival and regeneration) may be impregnated into an artificial bridging substrate such as Elvax®, or some other solid medium, and placed at or near the site in the body (such as a lesion) at which neuronal regeneration is required. Such impregnated substrates may also be used in vitro.

Gene therapy may also be employed to effect the endogenous production of the osteonectin protein or functional equivalent by the relevant cells in a subject. This aspect of the invention provides a method for stimulating the regeneration of a neuronal cell, wherein the neuronal cell is exposed to the osteonectin protein or functional equivalent through the introduction into a patient of a nucleic acid molecule that expresses the osteonectin protein or or functional equivalent thereof.

For a review of general methods of gene therapy, see e. g. Anderson, Science (1992) 256: 808-813; Nabel and Felgner (1993) TIBTECH 11: 211-217; Mitani and Caskey (1993) TIBTECH 11: 162-166; Mulligan (1993) Science 926-932; Dillon (1993) TIBTECH 11: 167-175; Miller (1992) Nature 357: 455-460; Van Brunt (1988) Biotechnology 6 (10): 1149-1154; Vigne (1995) Restorative Neurology and Neuroscience 8: 35-36; Kremer and Perricaudet (1995) British Medical Bulletin 51 (1) 31-44 : Haddada et al. (1995) in Current Topics in Microbiology and Immitnoloa Doerfler and Bohm (eds) Springer-Verlag, Heidelberg, Germany; and Yu et al., Gene Therapy (1994) 1: 13-26.

Gene therapy can occur in vivo or ex vivo. Ex vivo gene therapy requires the isolation and purification of patient cells, the introduction of a therapeutic gene and introduction of the genetically altered cells back into the patient. This aspect of the invention provides a method for stimulating the regeneration of a neuronal cell in a patient, wherein the neuronal cell is exposed to the osteonectin protein or a or functional equivalent thereof through the introduction into the patient of a cell that expresses the osteonectin protein or functional equivalent in a therapeutically-effective amount.

In contrast, in vivo gene therapy does not require isolation and purification of a patient's cells. Nucleic acid molecules, or vectors including such molecules, are applied directly to the patient. Such nucleic acid molecules may comprise the coding region for the osteonectin or functional equivalent. However, an alternative scenario involves the delivery of a nucleic acid molecule that induces the expression of the osteonectin protein or functional equivalent in vivo. For example, such a nucleic acid molecule may encode a transcription factor that specifically induces osteonectin expression.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or tissue.

Suspensions of transfected cells can also be injected using microneedles and deposited with stereotaxic precision in desired areas of the central nervous system. Cell therapy as a strategy is, for example, described in Nature, vol 392, supp, 30 Apr. 1998.

Gene guns or hyposprays may also be used for administration of gene therapy-based medicaments. Typically, therapeutic compositions may be prepared as injectables, either as liquid solutions or suspensions; solid forms suitable for solution in, or suspension in, liquid vehicles prior to injection may also be prepared. The compositions can also be administered into a lesion. Dosage treatment may be a single dose schedule or a multiple dose schedule.

Of course, to be effective therapeutically for central nervous system targets, an osteonectin-based drug desirably should be able to penetrate the blood-brain barrier when intravenously administrated. The intact central nervous system has a blood-brain barrier that severely limits entry of proteins into the brain or spinal cord if delivered into the blood stream. Many drugs do not readily penetrate into the brain. Drugs which are unable to penetrate the blood-brain barrier can be effectively administered by, for example, an intraventricular route of delivery. Such drugs can also be used to treat peripheral nerves where penetration of the blood-brain barrier is not an issue or in trauma and certain other diseases and disturbances of the brain or spinal cord, where the blood-brain barrier itself is damaged.

Typical preparations for administration include sterile aqueous or nonaqueous pharmaceutical compositions, including solutions, suspensions and emulsions. A pharmaceutical composition may contain a pharmaceutically acceptable carrier, for administration of the therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, humans are preferred subjects for treatment.

In addition, the invention also relates to a cell which produces osteonectin or a functional equivalent for use as a medicament per se, such as in the application discussed herein. Thus, the invention also encompasses a pharmaceutical preparation, which comprises a cell producing osteonectin or a functional equivalent thereof according to the invention together with a pharmaceutically acceptable carrier for use in gene or cell therapy methods. An especially advantageous method for treatment according to the invention is a method wherein osteonectin-producing cells are administered to a patient, and where the cells used were originally obtained from the actual individual receiving the therapy. Such cells may, for example, be fibroblasts originating from the patient. Thus, using this method, immunological rejection of non-autologous biological material can be circumvented.

The dosage and length of treatment with osteonectin or a functional equivalent depends on the disease state being treated. The duration of treatment may be a day, a week, or longer, and may, in the case of a chronic progressive illness, such as Alzheimer's disease, last for decades. The osteonectin should of course be administered in a therapeutically effective amount, that is, an amount effective to mediate the beneficial effects of osteonectin upon neuronal cell survival and regeneration. The precise effective amount for a human subject will depend upon the severity of the disease state, general health of the subject, age, weight, and gender of the subject, diet, time and frequency of administration, drug combination(s), reaction sensitivities, and tolerance/response to therapy.

This amount can be determined by routine experimentation and is within the judgment of the clinician. Generally, an effective dose will be from 0.01 mg/kg to 50 mg/kg, preferably 0.05 mg/kg to 10 mg/kg. However, the effective dose may be even higher than this if the osteonectin protein or functional equivalent is introduced in concentrated form at the site in the body at which neuronal regeneration is required.

In vitro in cell culture, the osteonectin protein or functional equivalent may be exposed to neuronal cells at amounts between 10 pg/ml and 10 μg/ml, preferably between 0.1 ng/ml and 100 ng/ml, more preferably between 0.5 ng/ml and 10 ng/ml.

Optionally, a neuronal cell may be exposed to the osteonectin protein or functional equivalent in the presence of one or more additional neurotrophic factors. Such neurotrophic factors include nerve growth factor, glial derived growth factor, brain derived growth factor, ciliary neurotrophic factor and neurotropin-3, vascular endothelial growth factor (VEGF), fibroblast growth factor (FGF) and platelet-derived growth factor (PDGF). Such neurotrophic factors can be administered by any means known in the art, as discussed above.

The invention also includes the use of the osteonectin protein or a fragment or variant thereof in the manufacture of a medicament for the stimulation of neuronal cell regeneration. Preferably, the medicament is for the stimulation of CNS neuronal cell regeneration.

There is speculation that osteonectin mediates many of its effects via an as yet unidentified receptor. Certainly the different effects of osteonectin can be dissected out in terms of intracellular signalling, with anti-proliferative effects dependent on G-protein coupled signalling and anti-adhesive effects via tyrosine kinase activation (Motamed and Sage, 1998). It is thus likely that osteonectin can have direct signalling roles, which may also, in part, explain its likely role in neuronal regeneration. Certainly the fact that osteonectin is taken up into RGCs from Schwann cell conditioned medium supports the notion of a specific receptor.

Accordingly, screens are currently on-going that are focused on the identification of the osteonectin receptor(s). Osteonectin protein may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells such as neurons, particularly sympathetic neurons, RGCs, PC12 cells, fibroblasts, cell membranes, cell supernatants, tissue extracts, or bodily fluids, or a population of cells into which a library of candidate receptors have been transformed, transfected or transduced). Co-immunoprecipitation of osteonectin with a membrane fraction of responsive cells (such as RGCs) is one preferred methodology in this respect.

This aspect of the invention thus provides a method of screening for a receptor protein that mediates the stimulation of neuronal cell regeneration, said method comprising exposing a cell to osteonectin or a functional equivalent, cross-linking the osteonectin protein or functional equivalent to a receptor on the cell, and analyzing the complex to identify the receptor. The efficacy of binding may be measured using biophysical techniques such as surface plasmon resonance and spectroscopy. Binding assays may be used for the purification and cloning of the receptor, but may also identify agonists and antagonists of the osteonectin protein, that compete with the binding of the osteonectin protein to its receptor. Standard methods for conducting screening assays are well understood in the art.

This aspect of the invention also includes methods that screen candidate cells for expression of the osteonectin receptor. Such a method may comprise contacting a candidate cell or a library of candidate cells with the osteonectin protein or a fragment or variant thereof and assessing the ability of the cell to effect stimulation of neuronal cell regeneration. In one embodiment, the library of candidate cells may be transformed, transfected, or transduced with a library of nucleic acid molecules, each nucleic acid molecule encoding a candidate receptor for osteonectin. In this manner, a functional screen may be utilized to screen for cells that express a receptor for osteonectin.

As will be apparent to the skilled addressee, any compound that shares the structure of the osteonectin active site (the binding site for its receptor(s) will be able to mimic the biological effects of osteonectin in stimulating the regeneration of neuronal cells.

Libraries of compounds that mimic the active site of osteonectin and thus share its advantageous properties may be identified in any one of a variety of drug screening techniques. Compounds for screening may be isolated from, for example, cells, cell-free preparations, chemical libraries or natural product mixtures. These agonists or antagonists may be natural or modified substrates, ligands, enzymes, receptors small organic molecules, peptides, polypeptides, antibodies and structural or functional mimetics. For a suitable review of such screening techniques, see Coligan et al., Current Protocols in Immunology 1(2):Chapter 5 (1991).

Suitable screening techniques will generally involve those based on binding properties, for example, using as bait an receptor molecule or antibody that binds to the active site of osteonectin and which can thus bind to compounds that are structural mimics of the osteonectin active site. Such bait compounds may be free in solution, affixed to a solid support, borne on a cell surface or located intracellularly. In general, such screening procedures may involve using appropriate cells or cell membranes that express osteonectin or the osteonectin receptor that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the osteonectin protein or receptor, using an appropriate detection system. Inhibitors of activation are generally assayed in the presence of a known agonist and the effect on activation by the agonist in the presence of the test compound is observed.

Alternatively, simple binding assays may be used, in which the adherence of a test compound to a surface bearing the osteonectin protein or receptor is detected by means of a label that is directly or indirectly associated with the test compound or in an assay involving competition with a labelled competitor. In another embodiment, competitive drug screening assays may be used, in which neutralising antibodies that are capable of binding the osteonectin protein or receptor specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the osteonectin protein in cells. For example, an ELISA may be constructed that measures secreted or cell-associated levels of osteonectin protein using monoclonal or polyclonal antibodies by standard methods known in the art, and this can be used to search for compounds that may inhibit or enhance the production of the osteonectin protein from cells or in tissues.

Another technique for drug screening which may be used provides for high throughput screening of compounds having suitable binding affinity to the osteonectin protein or receptor. In methods of this type, large numbers of different small test compounds are synthesised on a solid substrate, which may then be reacted with the osteonectin protein or receptor and washed. One way of immobilising the protein or receptor is to use non-neutralising antibodies. Bound polypeptide may then be detected using methods that are well known in the art. Purified polypeptide can also be coated directly onto plates for use in the aforementioned drug screening techniques.

The invention also includes a screening kit useful in the methods for identifying compounds of the nature described above.

The invention also includes compounds identified using the screening methods and kits of the above-described aspects of the invention. Such compounds may be, for example, agonists, antagonists, ligands, receptors, substrates and enzymes, and other compounds which mimic the activity of the osteonectin protein that are discovered by the methods that are described above.

It is considered that in silico methods are also of immense utility in the elucidation of the structure of the active site of the osteonectin protein as well as in the search for the osteonectin receptor(s) that are potentially responsible for mediating the effects on neuronal cell regeneration that have been observed for osteonectin. For example, numerous methods are now available for homology modeling. Examples include the POCKET program (Levitt, D. G. and Banaszak, L. J., J. Mol. Graphics 1992, 10, 229-234), the GRID potential (Goodford, P. J. 1985, J. Med. Chem. 28,849-857) the DOCK program (Kuntz, I. D., Blaney, J. M., Oatley, S. J., Langridge, R. & Ferrin, T. E., J. Mol. Biol. 1982, 161, 269-288) and the

MSI Ludi program (Bohm, H. J., J. Comput. Aided Mol. Des. 1992, 6, 61-78). By comparing the sequence of osteonectin with other proteins of similar sequence and ideally of known structure, it is anticipated that the active site of the osteonectin protein that is responsible for the activity documented herein may be identified. This will then pave the way for the identification of agents that mimic the active site of the osteonectin protein.

Various aspects and embodiments of the present invention will now be described in more detail by way of example, with particular reference to the osteonectin protein. It will be appreciated that modification of detail may be made without departing from the scope of the invention.

### BRIEF DESCRIPTION OF THE FIGURES

FIG. 1: Summary for the protocol to isolate Schwann cell factors

FIG. 2: Fluorography of primary Schwann cell medium (SCM) and RGC uptake. Lane 1—SCM. Lane 2—SCM after incubation with RGCs. Lane 3—RGC lysate after incubation with SCM. Lanes 4-7—Control lanes of RGC lysates following incubation with radiolabelled medium with (lanes 4 and 6) or without spin column step (lanes 5 and 7) and with (lanes 4 and 5) or without (lanes 6 and 7) cycloheximide treatment to show that the 41 kDa band in lane 3 does derive from the SCM and not any endogenous protein synthesis in RGCs. Note that endogenous RGC protein synthesis is all but eliminated where both the spin column and cycloheximide are employed, though some is still just apparent. In later experiments a high level of cold cysteine and methionine was included in the uptake assay to further reduce endogenous synthesis of radiolabelled proteins.

FIG. 3: A—Fluorography of Schwann cell secreted proteins. Lane 1—radiolabelled primary SCM (SCM*) prior to passing through the spin column; lane 2—SCM* after passing through the spin column (note that this procedure does not affect the protein make up of the SCM*); lanes 3 and 4—SCM* after incubation with retinal ganglion cells (different preparations); lane 5 and 6—retinal ganglion cell (RGC) lysates, after 24 hr incubation with SCM*, showing a number of radiolabelled bands taken up from the SCM* (indicated by arrows; approximate molecular weights in kDa are shown); modified from Bampton, 1998. B—Graph shows a typical plot of log (molecular weight; Mr) versus relative mobility (distance migrated/gel front) of the standard proteins. From the equation of the regression line an estimate of the molecular weights of the internalised proteins can be made.

FIG. 4: Repeats of the radiolabelling uptake assay. Lane 1—SCM*; lane 2 SCM* after incubation with RGCs; lane 3—RGC lysate; lane 4 (B only) RGC lysate after longer exposure. Although there is a slight decrease in general radiolabel intensity after incubation with RGCs (presumably reflecting a loss through some non-specific interactions with the RGCs), in B there are two bands, at 4 kDa and 32 kDa (arrowed) with a large intensity decrease that is reflected by an appearance in the RGC lysate lane.

FIG. 5: Radiolabelling uptake assay, using whole dissociated retina. The bands seen with RGCs alone, notably that in the 40-42 kDa region, are still present, but several others are also apparent (notably at 39 kDa and 18 kDa) which may indicate signalling to other retinal cell types.

FIG. 6: Autoradiographs of 2-D analysis of radiolabelled Schwann cell secreted protein uptake by RGCs. A—Radiolabelled SCM (SCM*); B—SCM* after incubation showing clear reduction in protein content; C—RGC lysate after 8 hour incubation with SCM*. The arrowed protein is decreased following incubation with RGCs and appears in the RGC lysate sample, indicating uptake from the medium. D shows an RGC lysate following incubation with radiolabelled fibroblast conditioned medium (CM)—this does not show uptake of the same protein, indicating the protein is specific to SCM*. Other proteins seen in this lysate, which also appear in the subsequent experiment (FIG. 7), are non-specific. Internal 2-D markers are 1—6.0-6.6/76 kDa (not visible here); 2—5.4-5.6/66 kDa; 3—5.0-5.1/43 kDa; 4—8.3-8.5/36 kDa (not visible here); 5—5.9-6.0/3 kDa; 6—4.5/21.5 kDa. 1-D markers to the sides are, from top to bottom, 205 kDa, 116 kDa, 97.4 kDa, 66 kDa, 45 kDa and 29 kDa.

FIG. 7: Repeat of radiolabelling uptake assay. A—C are equivalent to conditions in FIG. 6, the arrow again indicating the protein taken up by RGCs. D shows primary SCM after RGC incubation with cold CM added with less loss of protein than without cold CM (B) and E the loss of uptake of this protein by RGCs (seen faintly in C). The other proteins that are seen in both C and E (circled) are not blocked by cold CM and may reflect some residual intrinsic RGC protein synthesis. Markers as in FIG. 6.

FIG. 8: 2-D electrophoresis of 10 μg samples of conditioned media. A —Internal 2-D markers alone. B—Primary SCM; C—Forskolin-Expanded SCM; D—Fibroblast CM; E—SCTM41 CM; F—PVGSCSV40T CM. This shows good separation in 2 dimensions and clear differences in the patterns obtained, indicative of distinct profiles of secreted of proteins, as already suggested in the 1-D analysis. Markers (numbered in A and B) as in FIG. 6.

FIG. 9A: Greyscale version of Z3 image analysis of primary SCM compared to other conditioned media—forskolin expanded SCM (A), fibroblast CM (B), SCTM41 CM (C) and PVGSCSV40T CM (D). Areas of overlap are black. Positions of the markers are shown in A and are as for FIG. 6. The arrowed spot in all 4 images is unique to the primary SCM and corresponds to that seen in the 2-D analysis of radiolabelled protein uptake by RGCs (see FIGS. 6 and 7). The spots indicated with triangular markers in images A, C and D show other proteins seen in all Schwann cell CM, except the SCTM41 CM. These may correlate with the differing biological effects of these media on PC12 cells compared to the SCTM41 CM. Markers as in FIG. 6.

FIG. 9B: Colour version of Z3 image analysis of primary SCM (shown in green) compared to other conditioned media (shown in pink)—forskolin expanded SCM (A), fibroblast CM (B), SCTM41 CM (C) and PVGSCSV40T CM (D). Areas of overlap are black. Positions of the markers are shown in A and are as for FIG. 6. The arrowed spot in all 4 images is unique to the primary SCM and corresponds to that seen in the 2-D analysis of radiolabelled protein uptake by RGCs (see FIGS. 6 and 7). The spots indicated with triangular markers in images A, C and D show other proteins seen in all Schwann cell CM, except the SCTM41 CM. These may correlate with the differing biological effects of these media on PCI2 cells compared to the SCTM41 CM. Markers as in FIG. 6.

FIG. 10: 100 μg conditioned media run on a two-dimensional gel and blotted onto PVDF membrane and Coomassie Blue stained. As can be seen, the system was able to cope with relatively high levels of protein, beyond the recommended amount of 10 μg. A—100 μg PVGSCSV40T CM run as a test. B—100 μg primary SCM; this particular sample contained high levels of material around 60-70 kDa, possibly from the use of some material from cells kept only two days in culture before transfer to serum free conditions. The arrowed spot, which matched the pI and molecular weight requirements, was taken for sequencing.

FIG. 11: Western blot showing control lanes loaded with purified bovine osteonectin running at around 42 kDa and concentrated primary rat SCM. A second band apparent and matched by a faint band in the 50 ng bovine osteonectin is present, perhaps indicative of a degradation product or differing glycoforms.

FIG. 12: Images of control F12 cultured postnatal day 4 rat retinal ganglion cells (A) and those grown with 0.5 ng/ml osteonectin (B).

FIG. 13: Neuron Survival and Axogenesis in P0 Sympathetic Neurons. A: Graphical representation of observed axonal outgrowth. B: P0 Sympathetic Neurons in different culture media at 48 hours (phase contrast). A. Control; B. NGF30 ng/ml; C. NT-3 50 ng/ml; D. PUG; E. RSC; F. RSC+NGF30 ng/ml; G. SCTM41; H. SCTM41+NGF30 ng/ml =50 μm.

FIG. 14: Effect of Trk Inhibitors on Axonal Outgrowth in P0 Ms SCG.

FIG. 15: Optimisation of the level of osteonectin in SCG cultures. SGCs were grown under control conditions with 10 ng/ml NGF and then osteonectin added in increasing doses.

FIG. 16: Results of an experiment in which P4 mouse sympathetic neurons were cultured in medium with 10 ng/mi NGF at 5000/per well, in absence A) or presence B) of 0.5 ng/ml osteonectin.

FIG. 17: FIG. 17A provides a graph showing the quantification of the effects of the optimal dose of 0.5 ng/ml of osteonectin in cultures of sympathetic neurons at varying densities, grown in the presence of 10 ng/ml NGF. FIG. 17B shows the effects of osteonectin on the number of neurites per neuron.

### EXAMPLES

### Example 1

Initial studies

RT-PCR analysis of neurotrophic factor expression was first performed. mRNA was extracted from P1 rat brain, P4 rat sciatic nerve (the source for fibroblast, primary SC cultures and the forskolin expanded SC cultures), sciatic nerve fibroblasts, primary SCs, forskolin SCs and the two clonal cell lines, SCTM41 and PVGSCSV40T. This revealed the expression of a wide range of neurotrophic molecules in the tissues and cells examined (see Table 1).

TABLE 1
Summary of RT-PCR. Although not strictly quantitative, notably low expression is
indicated.

| | | Müller | Sciatic | | Primary | Forsk. | | PVGSC- |
|---|---|---|---|---|---|---|---|---|
| Factor | Brain | Glia | Nerve | Fibroblast | SC | SC | SCTM41 | SV40T |
| NGF | + | + | + | + | + | + | + | + |
| BDNF | + | + | + | + | + | + | + | + |
| | | | (low) | | | | | |
| NT-3 | + | + | + | + | − | + (low) | − | − |
| NT-4/5 | + | + | + | + | + | + | +/− | + |
| CNTF | + | + | + | + (low) | + | + | + | + |
| GDNF | + | + | + | + | + | + | + | + |
| | | | (low) | | | | | |
| FGF-2 | + | + | + | + | + | + | + | + |

Abbreviations are as follows:
NGF—Nerve Growth Factor;
BDNF—Brain-Derived Neurotrophic Factor;
NT-3—Neurotrophin-3;
NT-4/5—Neurotrophin-4/5;
CNTF—Ciliary Neurotrophic Factor;
GDNF—Glial-Derived Neurotrophic Factor;
FGF-2—Fibroblast Growth Factor-2.

A similar study was then performed using Western Blot analysis (see Table 2).

TABLE 2
Summary of Western blot data on conditioned media.

| | Primary | Forsk. | SCTM41 | PVGSCSV40T | Fibroblast |
|---|---|---|---|---|---|
| Factor | SCM | SCM | CM | CM | CM |
| NGF | −* | − | +* | −* | −* |
| BDNF | +* | − | −* | −* | − |
| NT-3 | − | − | − | − | − |
| NT- | + | − | − | − | − |
| 4/5 | | | | | |
| CNTF | − | − | − | − | − |
| GDNF | − | − | − | − | − |
| FGF-2 | − | − | + | − | − |

*indicates where a band was detected at the appropriate molecular weight for the precursor form.

Hardly any significant levels of trophic factors were detected by this methodology.

These results showed that, although there may be widespread trophic factor expression in Schwann cells, there is little secretion of biologically significant levels of these factors. These data have clear implications for the widespread assumption in regeneration studies that such factors are those critical to the observed axonal extension (e.g. Dezawa and Adachi-Usami, 2000). Moreover, depending on the provenance of the Schwann cells, the expression and secretion of trophic factors is variable, again with implications for grafting studies using purified Schwann cells or cell lines.

To further examine the nature of the conditioned media from the various cell types, bioassays were performed using PC12 cells in culture as a model of a peripheral neuron and compared with the actions of known factors (data not shown).

The range of factors tested could not mimic the effects of conditioned media from the primary Schwann or PVGSCSV40T cells. The effects of the conditioned media did not fit with the profiles of any other factors known to affect normal PC12 cells, suggesting that potentially novel factors are of importance.

Bioassays were then performed using retinal ganglion cells as a model central neuron. The resulting data suggest that, as for the PC12 cells, as yet unidentified factors produced by Schwann cells are primarily responsible for the biological effects of the conditioned medium. Neither cell line produced cell medium able to mimic the effects of primary Schwann cell medium and forskolin expansion/long term culture also clearly diminished the efficacy of the Schwann cells' secretions.

Efforts were therefore directed to the identification of the critical secreted molecules by a radiolabelling assay.

### Example 2

Radiolabelling Experiments

The evidence from the experiments discussed in Example 1 above strongly suggests that unidentified, possibly novel, factors are responsible for the biological effects of primary Schwann cell conditioned medium on neurons, most notably on purified retinal ganglion cells (RGCs). In an attempt to identify these molecules, biochemical characterisation and comparison of conditioned media by protein electrophoresis was carried out. In addition a radiolabelling study was employed to identify the most likely candidates for the observed biological effects.

Radiolabelling of proteins using 35-S labelled cysteine and methionine provides a sensitive method for the detection of low levels of protein. The methodology used here relies on the fact that, for much signalling in neurons, there is a need for uptake and transport of the signalling molecule back to the cell soma. Thus target-derived factors are taken up at the synapse and transported down the axon to the cell body, where they can exert their effects (Reynolds et al., 2000). Such transport is also a feature of retinal ganglion cells, occurring both retrogradely (DiStefano et al., 1992; Ehlers et al., 1995; von Bartheld et al., 1996b) and anterogradely (von Bartheld, 1996a) mediated by the cognate neurotrophin receptors and the p75 receptor (von Bartheld et al., 1996b).

In more general terms, the binding of a ligand to its receptor is often accompanied by receptor-ligand internalisation. The whole receptor-ligand complex may be targeted to endolytic compartments for degradation, thereby terminating the signal and decreasing the cell's responsiveness to further stimulation. Alternatively, only the ligand may be degraded and the receptor recycled to the cell surface, thereby terminating the original signal but maintaining the cell's responsiveness to further signalling (Crumpton et al., 1983). However, it is also apparent that signalling from the endosomal compartment can occur, suggesting that the ligand can be spared, at least for the time necessary for further signalling. Thus in non-neuronal cells the activation of different signalling pathways by the epidermal growth factor receptor is dependent on whether it is localised to the cell surface or endosome (Carpenter, 2000). Another example is the activation of the Erk-1/2 MAP kinases, which requires endocytosis of G-protein coupled receptors (Luttrell et al., 1999).

Radiolabelling

The radiolabelling uptake assay used here aims to make use of these normal processes to identify molecules that neuronal cells bind and thereafter internalise.

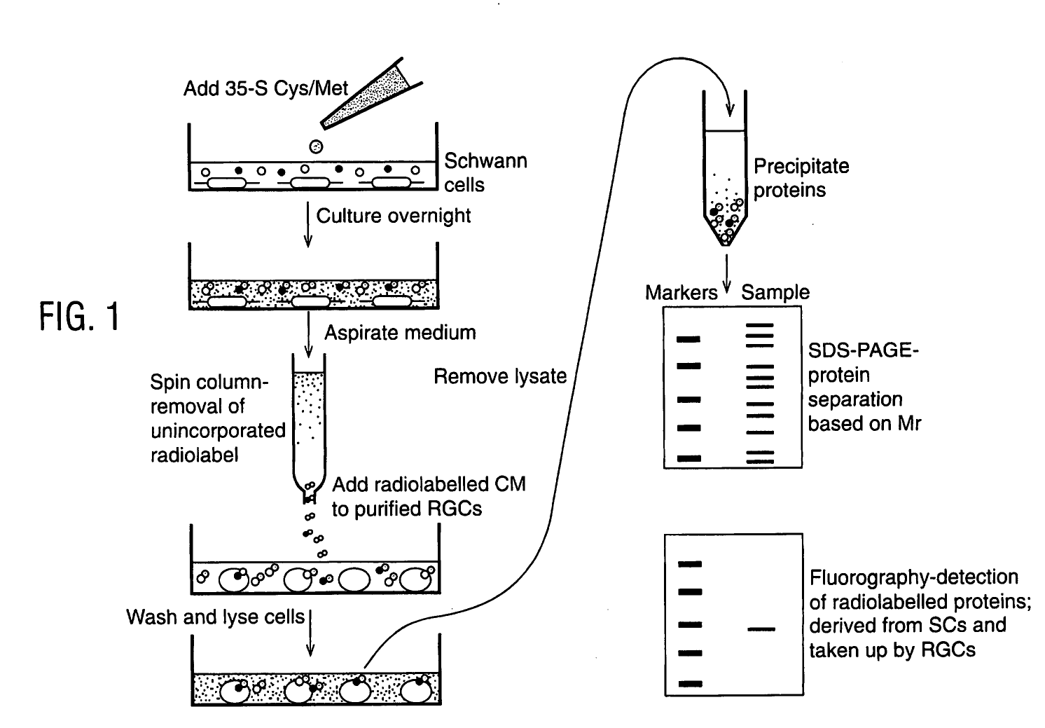

Schwann cell (both primary and cell line) and fibroblast (as negative control) cultures were incubated with 300 μCi Translabel™ (ICN) or EXPRES35S35 (NEN), containing 35^{s}-methionine and cysteine, to radiolabel secreted proteins. Unincorporated radiolabel was removed from the conditioned medium (CM) which was then fed to purified cultured neurons. These were incubated for 8 hours or overnight in the presence of protein synthesis blockers. The cells were lysed and analysed for protein uptake (Methodology summarised in FIG. 1).

Scintillation Counting

To ensure that the washing of the RGCs was efficiently removing residual radioactivity and non-specifically bound proteins, scintillation counts of one set of washes were made. 100 μl of wash (or radiolabelled conditioned medium for comparison) was added to 4 ml of scintillation fluid and counts per minute (cpm) determined using a Beckman scintillation counter. Total radioactivity per ml wash in MBq was calculated using the following equation:

$$Radioactivity MBq \text{/} ml = \frac{( cpm \times 10 )}{( 222 \; 000 )} \times 0.037$$

cpm is multiplied by 10 to give cpm/ml, which is divided by 222 000 to give radioactivity in microCuries; there are 37 MBq per milliCurie, so multiplication by 0.037 gives radioactivity in MBq per ml.
Removal of Radiolabel and Inhibition of RGC Protein Synthesis

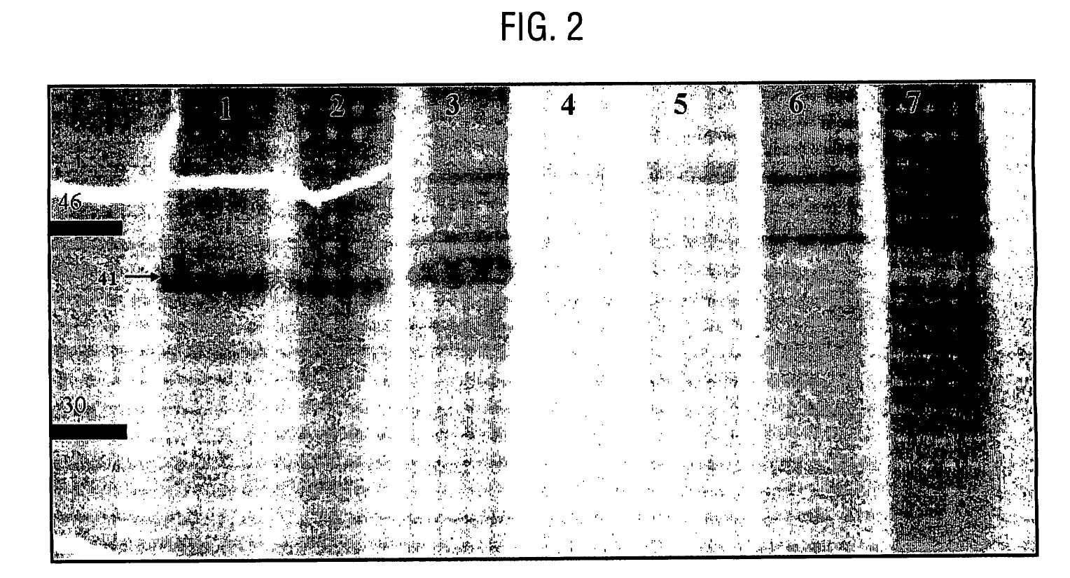

Of particular importance to this methodology was the demonstration that the proteins observed from retinal ganglion cell lysates were indeed taken up from the conditioned medium and not due to any residual endogenous synthesis. To this end a comparison was made between lysates from RGCs incubated with radiolabelled SCM and medium with radiolabel which had or had not been put through a spin column and with or without the addition of cycloheximide. This is shown in FIG. 2. Both the use of the spin column and cycloheximide reduce incorporation of radiolabel into the RGC proteins, the combination of both measures being most effective. However, a low level of residual synthesis was observed which was why, in later radiolabelling experiments, an excess of cold methionine and cysteine were included to try to out-compete the radiolabel in such residual synthesis.

Note in this experiment that the basic premise, that ligands used by the RGCs are intemalised, appears to be sound; in FIG. 2 a band can be seen appearing in the RGC lysate with an apparent decrease in that band intensity in the radiolabelled medium following incubation with the RGCs (lanes 1-3).

Radiolabelling Uptake Assay

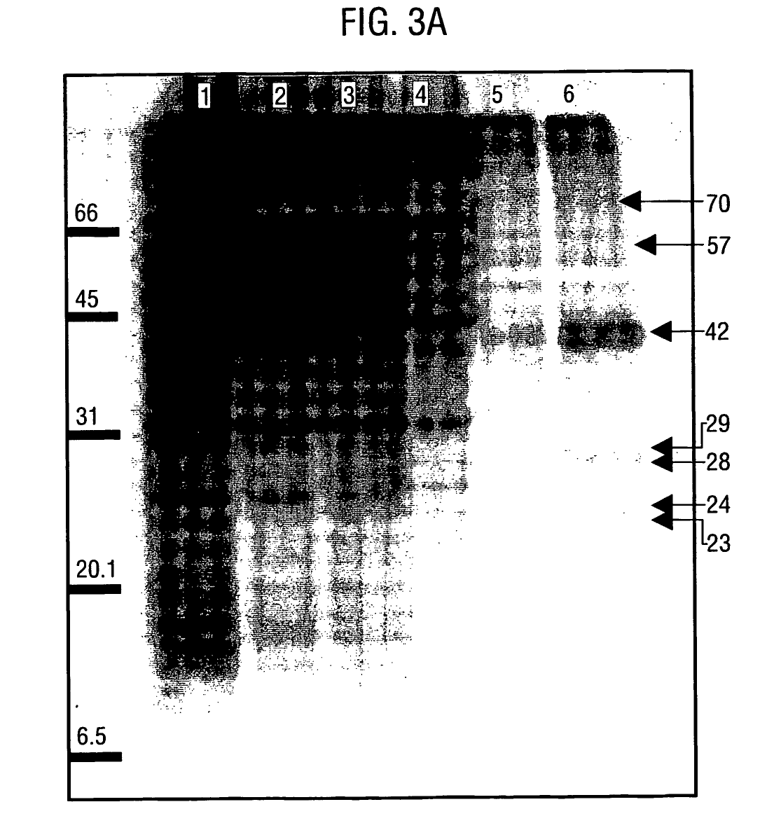

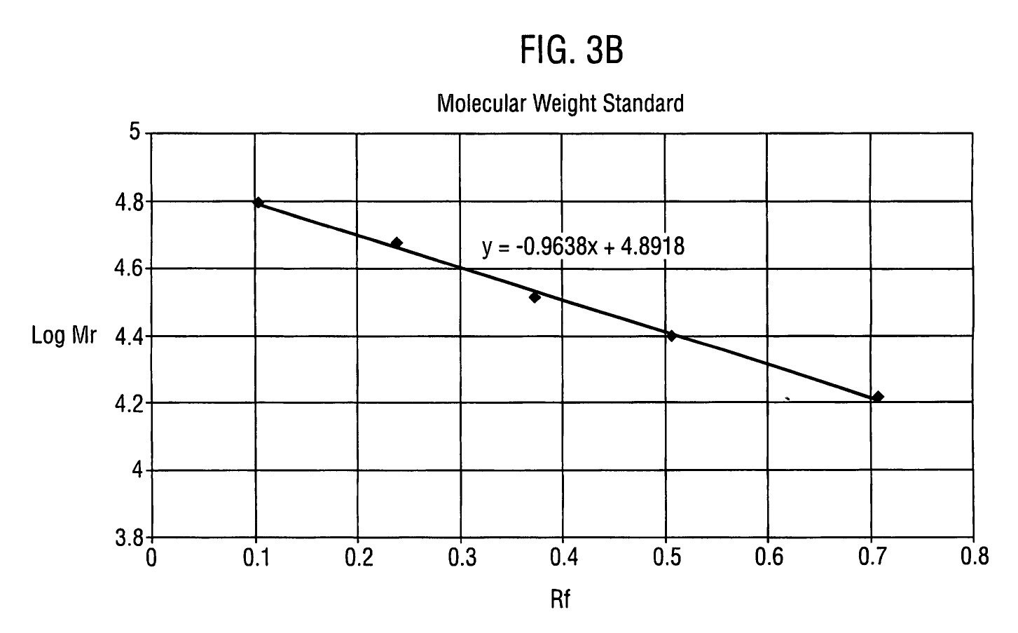

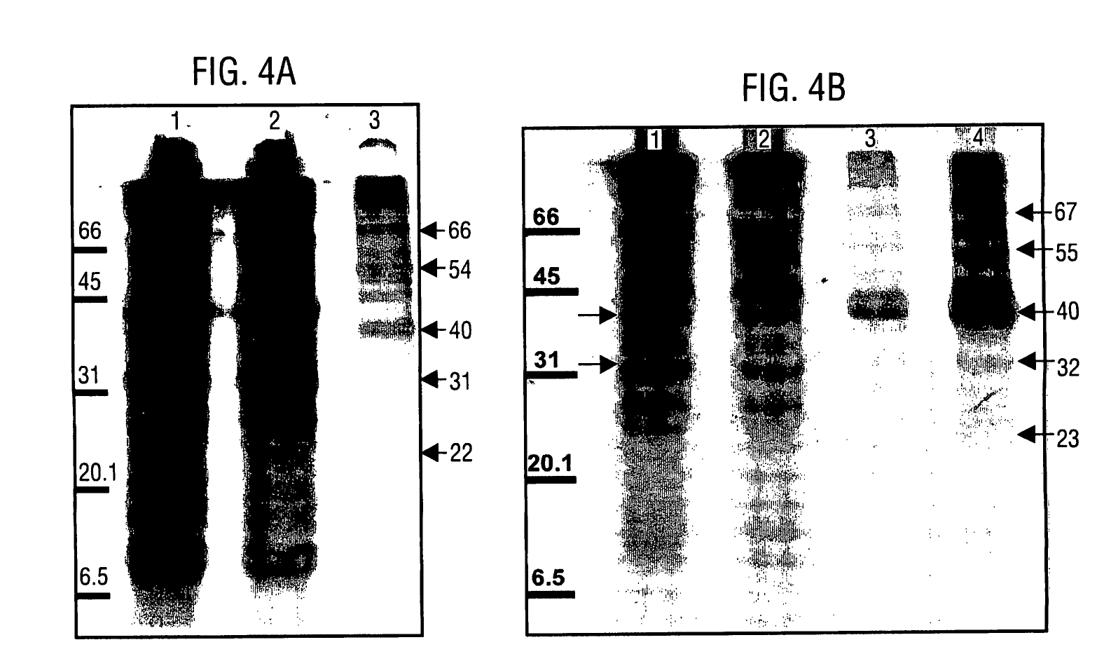

This was attempted in a variety of ways. Interestingly, no proteins could be clearly detected from lysates of PC12 cells incubated with any of the radiolabelled conditioned media. Neither were any proteins reliably seen in retinal ganglion cell (RGC) lysates incubated with SCTM41 CM, PVGSCSV40T CM or fibroblast CM, although this is less surprising given their lack of biological effect. However, incubation of radiolabelled primary SCM (SCM*) with RGCs resulted in reliable uptake of a range of proteins. Typical results are shown in FIG. 3 and 4.

A series of bands were identified in multiple repeats (n=5) of the radiolabelling study, using different Schwann cell and retinal ganglion cell cultures. The primary SCM is rich in secreted proteins, several of which appear to be taken up by the retinal ganglion cells, as shown FIGS. 3 and 4. The estimated molecular weights of proteins identified in 5 repeats of this experiment are summarised in table 3.

TABLE 3

| Exper- | Exper- | | | |
|---|---|---|---|---|
| iment 1 | iment 2a | Experiment 2b | Experiment 3 | Experiment 4 |
| | 70 kDa | 70 kDa | 66 kDa | 67 kDa |
| | 57 kDa | 57 kDa | 54 kDa | 55 kDa |
| 41 kDa | 42 kDa | 42 kDa | 40 kDa | 40 kDa |
| | | | 31 kDa | 32 kDa |
| | 29 kDa | 29 kDa | | |
| | 28 kDa | 28 kDa | | |
| | 24 kDa | 24 kDa | | |
| | 23 kDa | 23 kDa | 22 kDa | 23 kDa |
| Approximate molecular weights (in kiloDaltons; kDa) of bands identified in radiolabelling experiments (see FIGS. 3-4). Equivalent bands have been grouped in rows; | | | | |
| # note that SDS-PAGE has an approximate 10% error in molecular weight determination. In all cases the 40-42 kDa band (in bold) was the most intense and appeared in all experiments. | | | | |
| # The 66-70 kDa and 54-57 kDa bands are also common; it should be noted that in experiment 1 (see FIG. 2; methodological considerations) the number of retinal ganglion cells used was | | | | |
| # lower than in the other experiments which may account for the loss of lower intensity bands, which are not detected. The lower molecular weight bands were seen clearly in experiments 2 | | | | |
| # and 3 (which used the same radiolabelled SCM, but different retinal ganglion cell preparations - shown in FIG. 3) but where present, more faintly in the latter experiments, | | | | |
| # hence separate bands could not always be resolved. | | | | |

As shown in FIG. 3, the spin column does not appear to have affected the protein make-up of the SCM*, the same bands appearing in both lanes 3 and 4, an important methodological point, although the band intensity is decreased, indicative of some loss of material at this stage. No difference is obvious between lane 4 and lanes 5 and 6, which would be indicative of depletion of a given protein from the SCM* by the RGCs, though this is hard to interpret due to a general decrease in intensity, possibly through some non-specific interactions with the RGCs, then lost in the washing steps. However, several bands appear in the RGC lysate, indicating that radiolabelled proteins have been taken up by the RGCs. In subsequent experiments (FIG. 4) a generally similar pattern of uptake is observed. Most notable is the continued uptake of a protein in the 40-42 kDa range, especially strong in FIG. 4B. Also notable in FIG. 4B is that there is a distinct loss of protein from the SCM* lane following incubation with the RGCs which is reflected by uptake of those same proteins by the RGCs, something not clear in the previous two runs due to the intensity of radiolabelling.

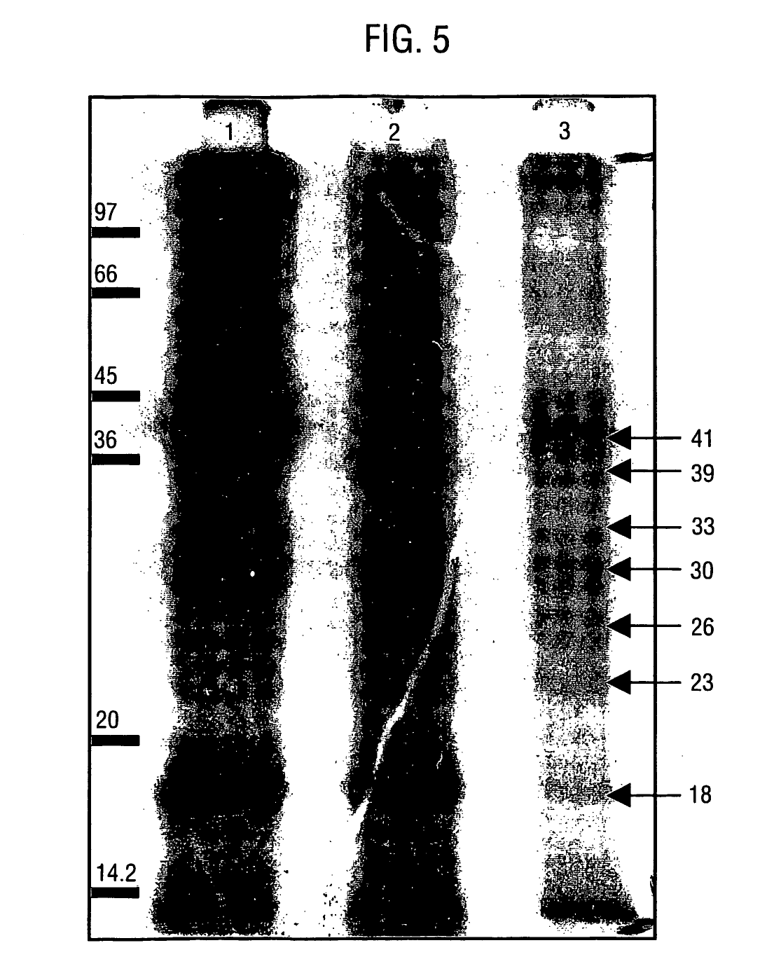

In one experiment, the whole dissociated retina was incubated with radiolabelled SCM, as shown in FIG. 5. Not all effects in vivo may be direct, but may be mediated via stimulation of other cell types (notably the Müller glia) so it was of interest to see how different the uptake pattern may be. Most of the bands taken up appear in the same molecular weight ranges seen previously, suggesting most effects may be directly on the neurons. However two did not fit with previous patterns using RGCs alone, at 39 kDa and 18 kDa. These may thus signal to, or via, other cells of the retina.

2-Dimensional Electrophoresis Studies

Beyond this, a comparative 2-dimensional electrophoretic analysis was carried out. By comparing the pattern of secreted proteins between conditioned media from different the Schwann cell preparations and correlating these to the biological effects already observed on PC12 cells and RGCs, candidate molecules for the survival and neuritogenic factors were identified.

Radiolabelling Uptake Assay

The medium conditioned by primary Schwann cells contains many secreted proteins. A simple comparison of both radiolabelled and cold conditioned media (data not shown) show that the range of proteins secreted by the different cell types studied was markedly different. It was therefore not always possible to accurately determine the proteins responsible; thus one could not always ascertain which of the proteins in the SCM* lanes had been taken up by the RGCs against the background of the other secreted proteins. Moreover a single band in a 1 -dimensional gel can contain several proteins of the same molecular weight.

Thus it was decided to repeat the radiolabelling experiment using a 2-dimensional gel analysis. This gives much greater separation of proteins, being based on both the isoelectric point and the molecular weight thereof, allowing much clearer identification. As a negative control, fibroblast conditioned medium was also employed. To ensure the results were specific to uptake of radiolabelled proteins, in one experiment an excess (50 μg) of cold primary SCM (from a dialysed and concentrated sample) was included to compete out binding of the radiolabelled protein.

SDS-PAGE and Fluorography

Proteins from 100 μl CM*, 150 μl CM* after incubation with neuronal cells (to allow for the additional 0.5 ml fresh medium added per 1 ml CM*) and 100 μl of cell lysate were precipitated and run on 1- or 2-D gels as previously described. After the run gels were fixed in 25% methanol (v/v)/5% acetic acid (v/v) and then soaked in a fluorographic solution (Amplify™; Amersham) for 25 minutes before being transferred onto a piece of Whatman 3 MM paper and overlaid with Saran wrap. The gel was placed on a gel dryer and dried at 80° C. for 2 hours, under vacuum. The dried gel was then placed in a light-proof cassette and a sheet of X-ray film (Hyperfilm™; Amersham) placed over the top and exposed overnight or for several days (or longer if necessary to obtain a good image) at −70° C. This is summarised in FIG. 1.

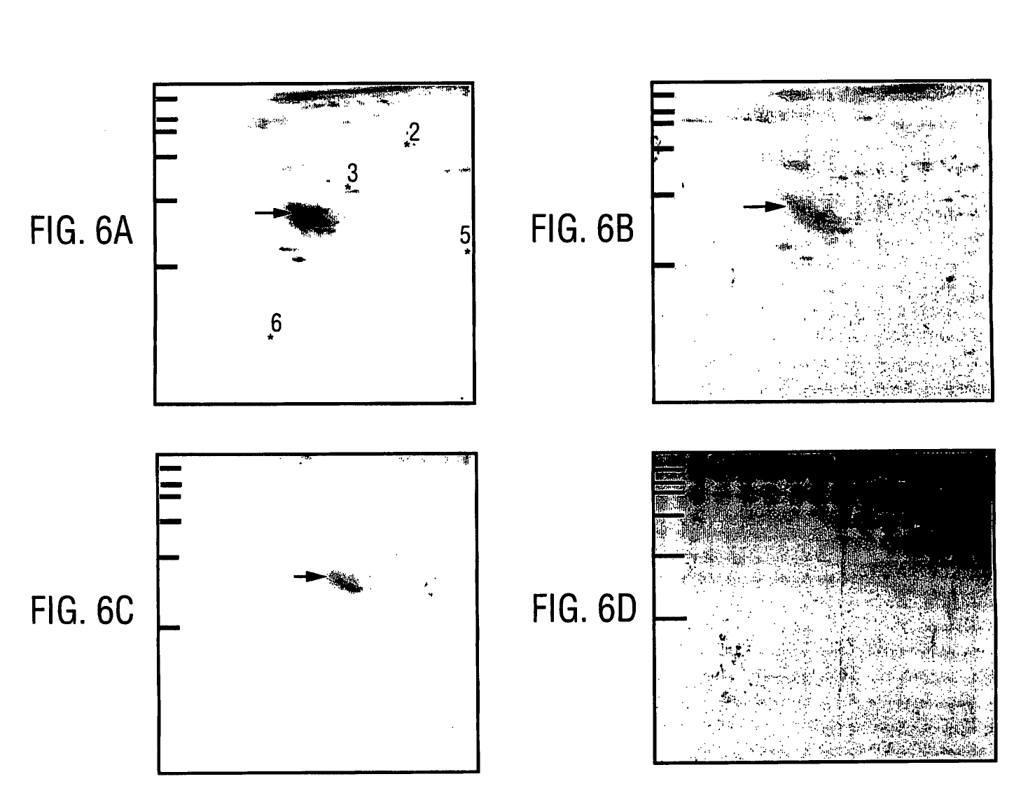

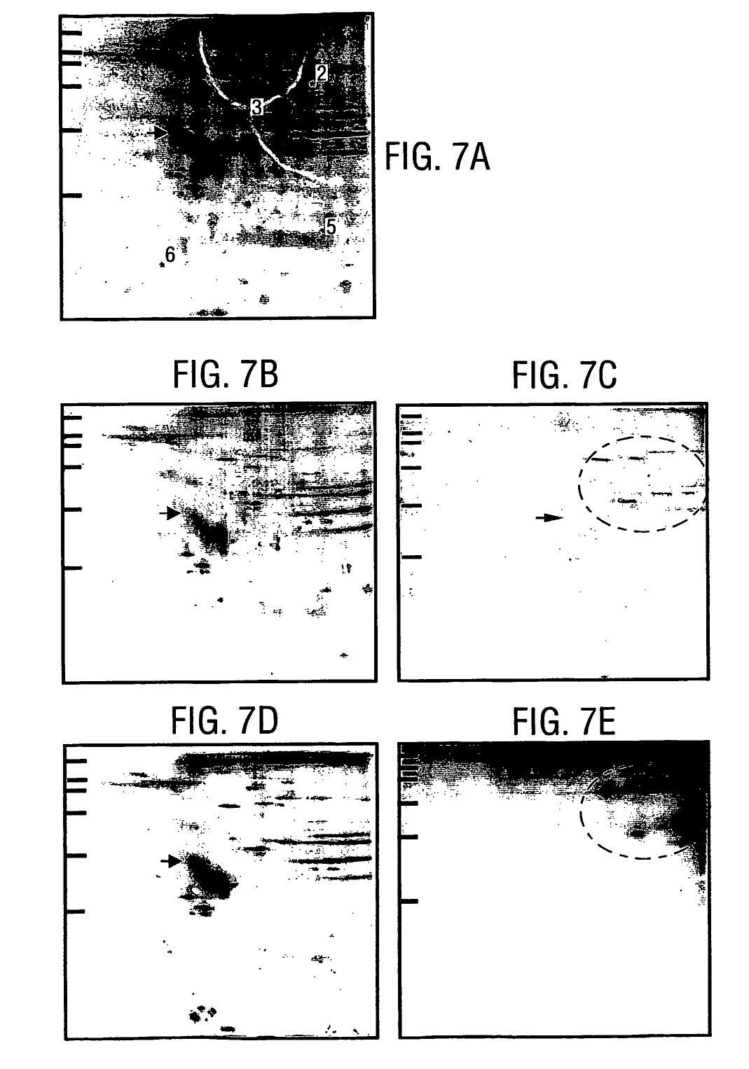

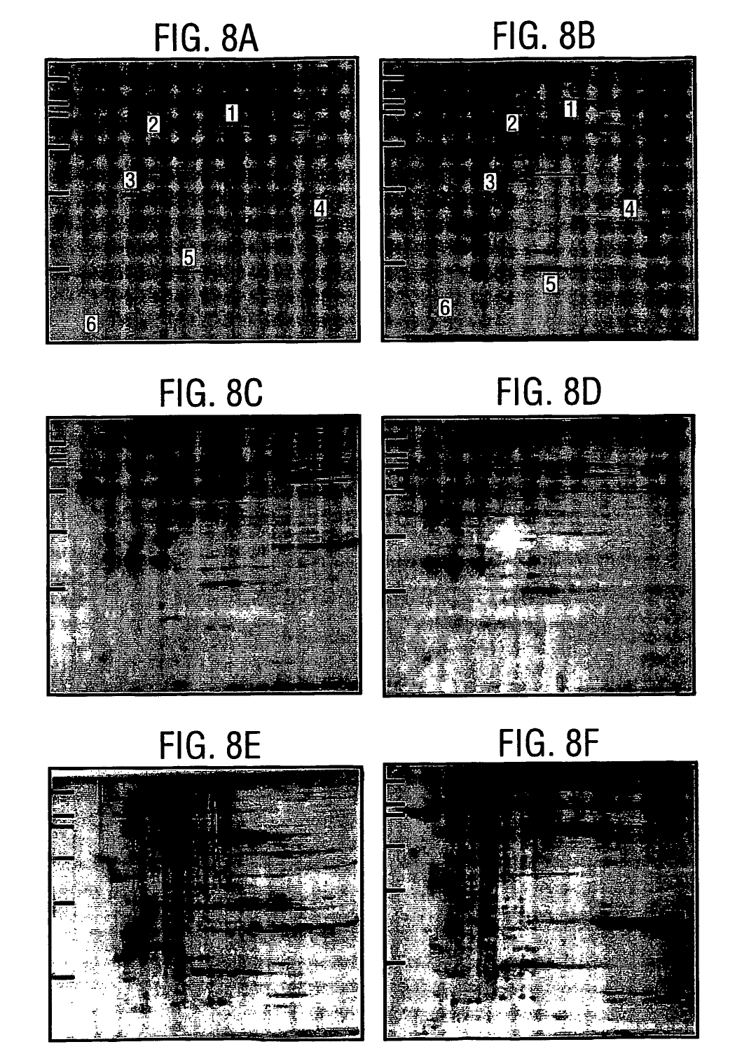

As seen in FIG. 6, there is a spot in the region of 40-42 kDa, consistent with the most commonly seen protein taken up in the 1-dimensional gel studies. From the position of the internal markers, one can estimate also that the protein has a pI between 4.5 and 5.0. No protein in this range is taken up from the fibroblast CM, so this is specific to the SCM. Several other proteins appear in the RGC lysate following incubation with radiolabelled fibroblast CM, but these are probably non-specific as they were also seen in the subsequent experiment (see FIG. 7 and below).

FIG. 7 shows a repeat of this experiment, but including one incubation with 50 μg dialsysed purified cold SCM. Faint uptake of the 40-42 kDa protein can be seen (fainter than the previous experiment because of a lower number of RGCs used) with concomitant loss of spot intensity in the radiolabelled SCM. Cold SCM competes out this spot, demonstrating it is not caused by stimulation of RGC protein synthesis despite inhibition thereof by cycloheximide. A series of other spots seen in this experiment were not competed out, suggesting they are not specifically taken up from the primary SCM. These spots are the same as those seen with fibroblast CM on RGCs and are thus most probably attributable to non-specific residual synthesis from the RGCs.

Comparative 2-D Analysis

Given the results from the radiolabelling study and the retinal ganglion cell bioassay, it was decided to compare the profile of secreted proteins from the various conditioned media in both one and two dimensions. A band and spot would be expected in the range seen in the radiolabelling study in the primary SCM but not in the other samples. This would correlate with the primary SCM being the only CM to show bioactivity on RGCs.

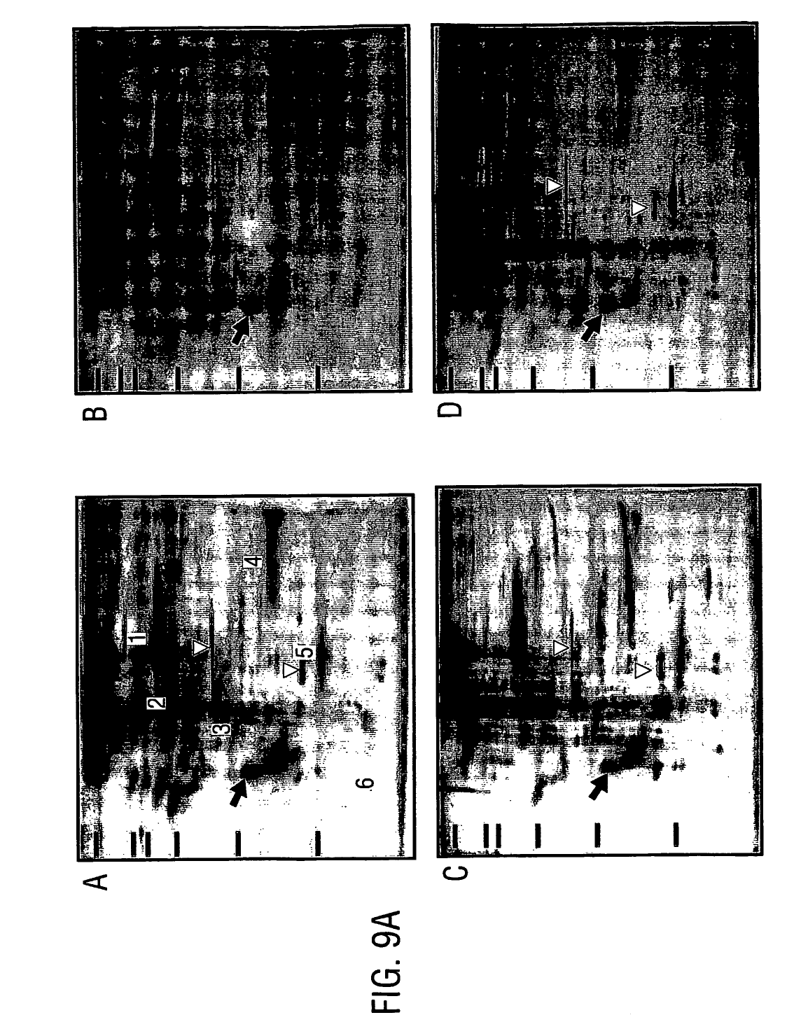

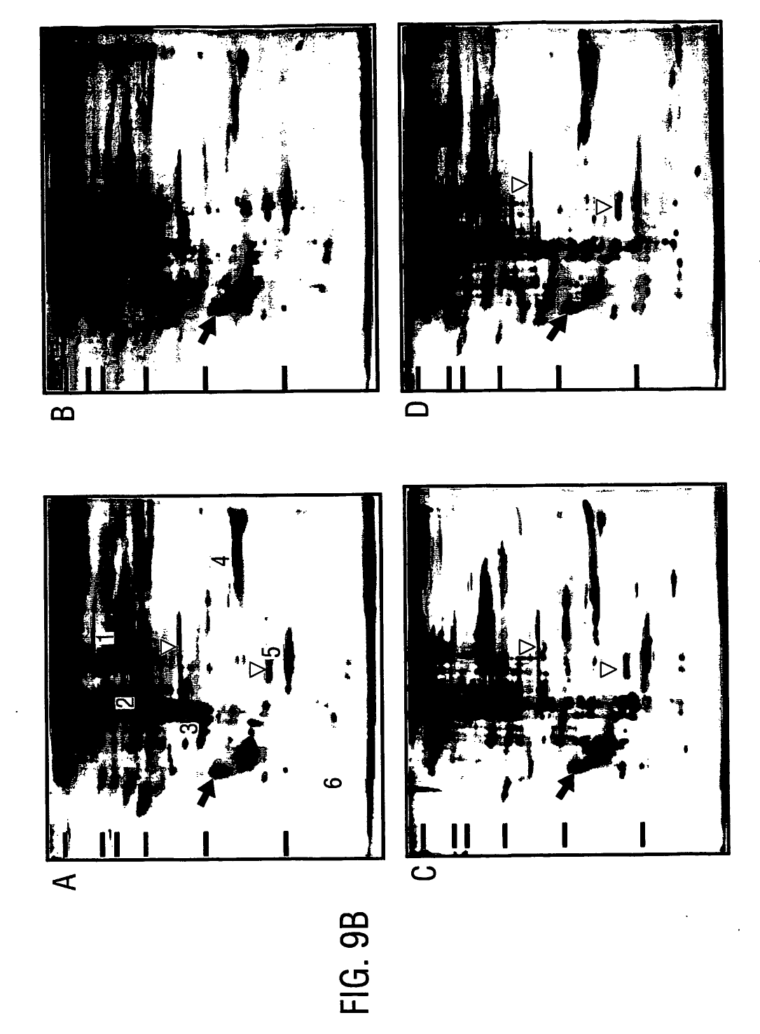

The silver 2-D gels can be seen in FIG. 8. These are one set of examples, but to ensure reproducibility, runs of primary SCM and PVGSCSV40T CM from different sets of cultures were also run, and found to give the same pattern. The 2-D gels were matched using the Z3 system (Compugen) and the data displayed in FIGS. 9A and 9B. A greyscale version (FIG. 9A) shows both sets of proteins in grey; areas of overlap are black. A colour version (FIG. 9B) shows one set of proteins coloured green (the primary SCM) and the other pink (for each of the other CM in turn); areas of overlap are black. As can be seen in FIG. 9A and 9B, a spot (indicated with an arrow in FIG. 9A; green coloured and also indicated with an arrow in FIG. 9B) in the molecular weight and pI range observed in the 2-D radiolabelling study is specific to the primary SCM and not seen in any other sample. This makes it the most likely candidate for the biological actions on the RGCs.

In addition, two proteins were noted that were expressed in three of the Schwann cell conditioned media, but not the SCTM41 CM. These may correlate with the bioactivity of these media on PC12 cell as compared to the SCTM41 CM. The first has a molecular weight of 57 kDa and pl in the region of 6.0 (running near the level of markers I and 5, pI6.0-6.6 and 5.9-6.0 respectively). The second has a molecular weight of 33 kDa and pI between 5.6 and 6.0 (the upper and lower ranges of markers 2 and 5 respectively, between which it lies).

Database Search

From the pI and molecular weight information it is possible to search the SWISS-PROT database using the Tagldent search tool (available under http://www.expasy.org), which compares inputted parameter with calculated parameters for the protein in the database. Any candidate molecules from the radiolabelling studies and 2-D comparative analyses were researched further in the literature.

Using Tagldent with input values of 41 000 Da±10% and pI4.75±0.25 and restricting the search to mammalia, 90 proteins were listed. The majority of these were clearly not of interest, with well-defined functions (e.g. complement C3 percursor), intracellular localisation both cytoplasmic (e.g. dual specificity phosphatase 6) and nuclear (e.g. transcription factors such as cyclic-amp-dependent transcription factor ATF-4) or membrane proteins (e.g. arsenical pump-driving ATPase).

However three proteins have well known neuronal roles. The low affinity nerve growth factor receptor p75 from mouse and human was noted and searching for the rat form (accession number P07174) gave parameters of pI4.45 and Mr 42 478 Da. The human form of syndecan-3 was also noted and a search for the rat form (accession number P33671) gave parameters of pI4.31 and Mr 41 413 Da. The mouse form of neuroserpin (accession number 035684), a serine protease inhibitor that inhibits plasminogen activators and plasmin, comes out with a pI of 4.69 and Mr of 44 588 Da.

A similar search for the differentially expressed proteins was also used. Input parameters of 33 000 Da±10% and pI5.75±0.25 yielded 174 hits, of which several had neuronal connections, namely neurotrimin (accession number Q62718), survival motor neuron protein (SMN; accession number 035876), neural proliferation and control protein precursor-1 (NPDC-1 protein; accession number Q64322) and syntaxin 4 (accession number Q08850). Literature searches suggest none of these as likely candidates.

Input values of 57 000 Da±10% and pI6.25±0.5 (necessarily broader, due to the streaked nature of this spot) yielded 571 hits, surprisingly few of which had a neuronal or growth factor connection. Of those hits of potential interest were the distintegrin and metalloproteinase domain (ADAM) family of proteins (notably AD11, accession number Q9R1V4, and AD21, accession number Q9JI76), which were further researched in the literature.

None of these was considered to be a likely candidate so efforts were directed towards sequencing the candidate protein.

Protein Sequencing

A simple database search, given the current lack of information on the actual running properties of many proteins in 2-D electrophoresis, was not able to pull out obvious candidate molecules. Therefore, efforts turned to actual identification of the 40-42 kDa protein seen in the radiolabelling uptake assay, as this methodology confirms its likely role.

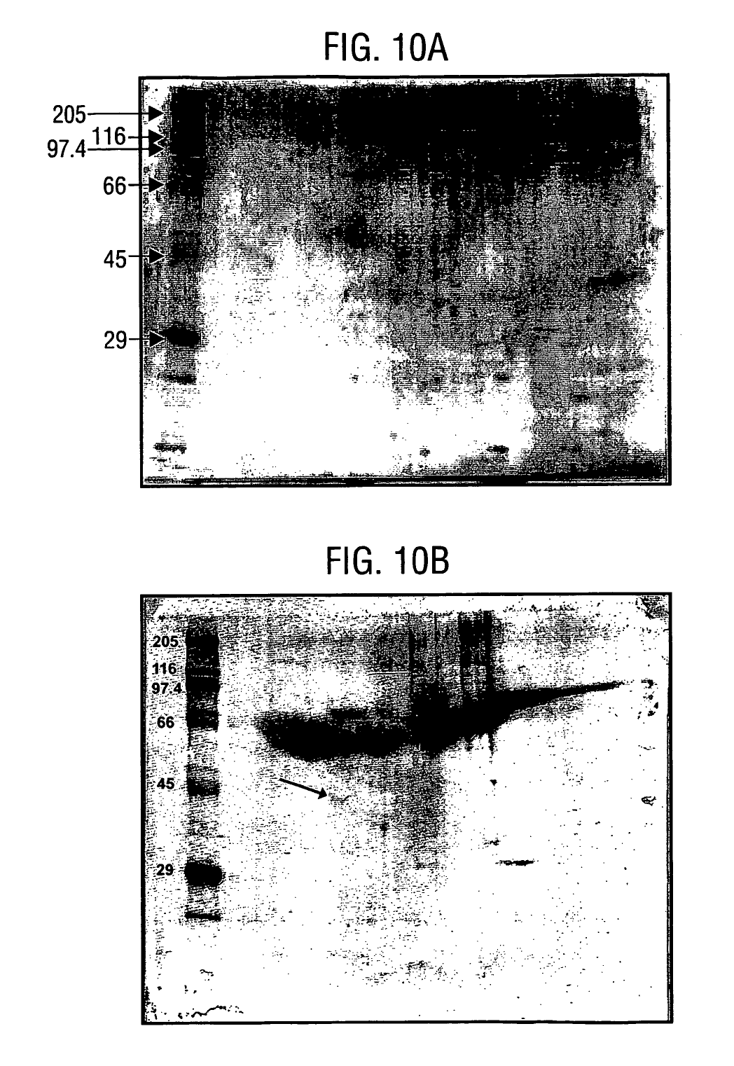

For successful sequencing by Edman degradation there is a limit of approximately 100 ng of protein required. Thus on a PVDF blot, the protein needs to be detectable by Coomassie Blue staining, the detection limit of which is about 100 ng. To maximise the amount of protein it was necessary to run more than the 10 μg recommended level for the first dimension in 2-D electrophoresis. To test the limits to which the system could be pushed higher levels (100 μg) of concentrated PVGSCSV40T CM were run, transferred to PVDF membrane and Coomassie Blue stained. As can be seen in FIG. 10, the system coped admirably with this higher level of protein.

Thus 100 μg of concentrated primary SCM was run, transferred to PVDF and Coomassie Blue stained; this is also shown in FIG. 10. A high level of protein around 60-70 kDa is clearly visible; this probably reflects the use of some conditioned medium from Schwann cells after only 2 days in culture (as opposed to the usual 4 days when collection was started) as this may well contain proteins secreted immediately following injury which are downregulated soon after. Nonetheless, a protein at the molecular weight and isoelectric point of interest was faintly visible and this blot was sent over to Tony Willis in the Department of Biochemistry, Oxford, for Edman sequencing. Despite the low level of protein (just above 1 picomole) the sequencing was successful and clear. The following sequence, using the one letter amino acid code, was obtained:-
APQTEAAEEMVAEET
   Where A=alanine, P=proline, Q=glutamine, T=threonine, E=glutamate, M=methionine and V=valine.

Searching the protein database by way of a BLAST search (which compares the sequence obtained with known sequences in the current SWISS-PROT database), the sequence shows 100% homology with the N-terminal domain of SPARC precursor from rat (secreted protein acidic and rich in cysteine), also known as osteonectin or basement membrane protein (BM-40).

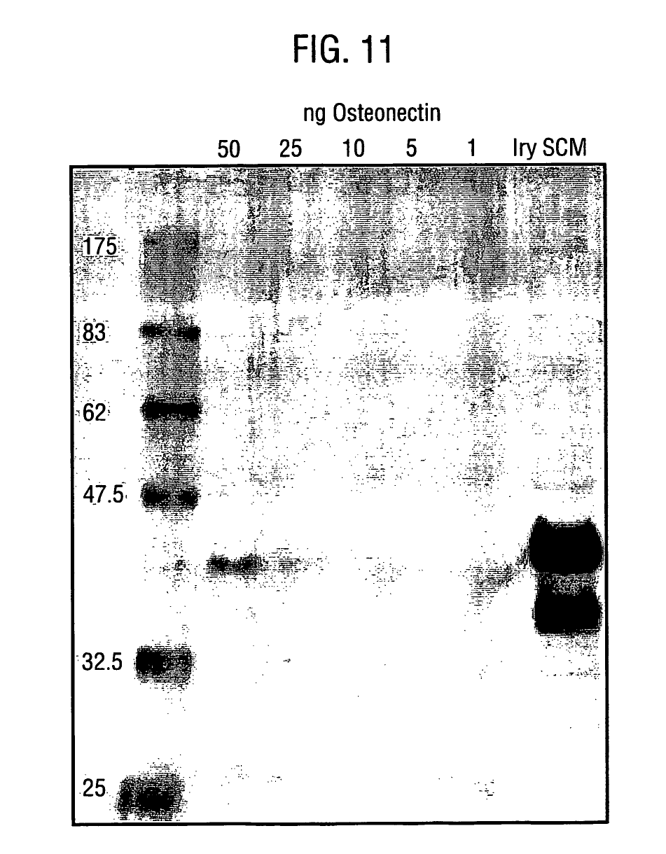

FIG. 11 shows a Western blot comparing purified bovine osteonectin to concentrated primary rat SCM. As can be seen from the gel, the purified bovine osteonectin runs at the same molecular weight as the osteonectin protein present in the concentrated primary rat SCM. A second band is apparent and is matched by a faint band in the 50 ng bovine osteonectin lane, perhaps indicative of a degradation product or differing glycoforms.

### Example 3

Effect of Osteonectin Protein on Neuronal Survival and Regeneration

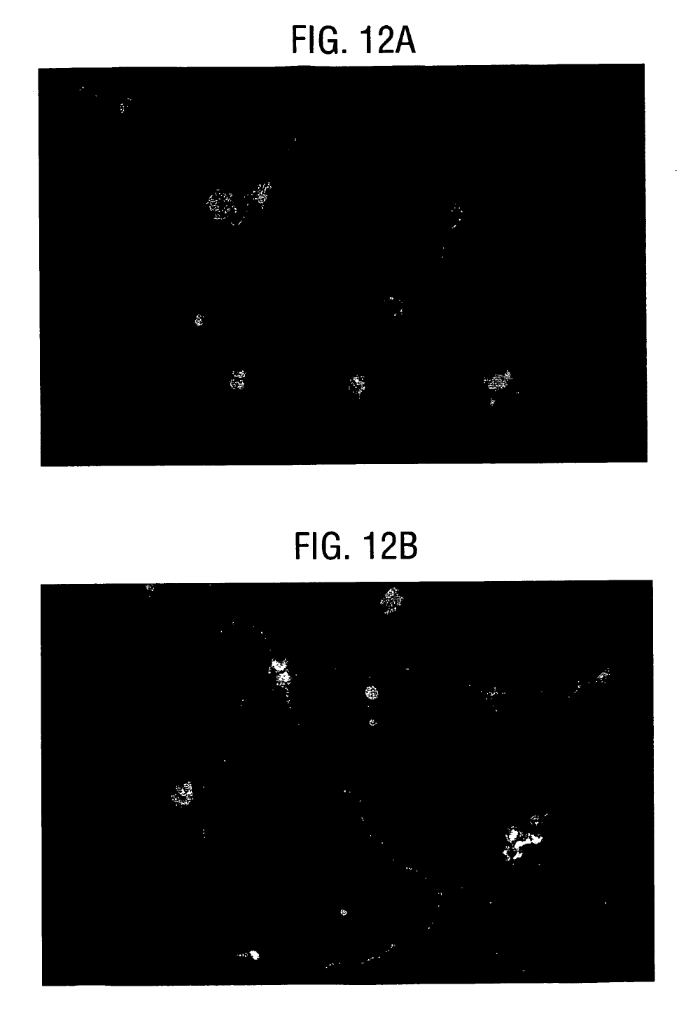

Images of control F12 cultured postnatal day 4 rat retinal ganglion cells are shown in FIG. 12A. The same cells grown in the presence of 0.5 ng/ml osteonectin are shown in FIG. 12B. It is apparent from FIG. 12B that significant stimulation of neuronal survival and regeneration occurs in the presence of the osteonectin protein in the culture media.

### Example 4

Effects of Schwann Cell Conditioned Medium on Sympathetic Neuron Survival and Axon Regeneration

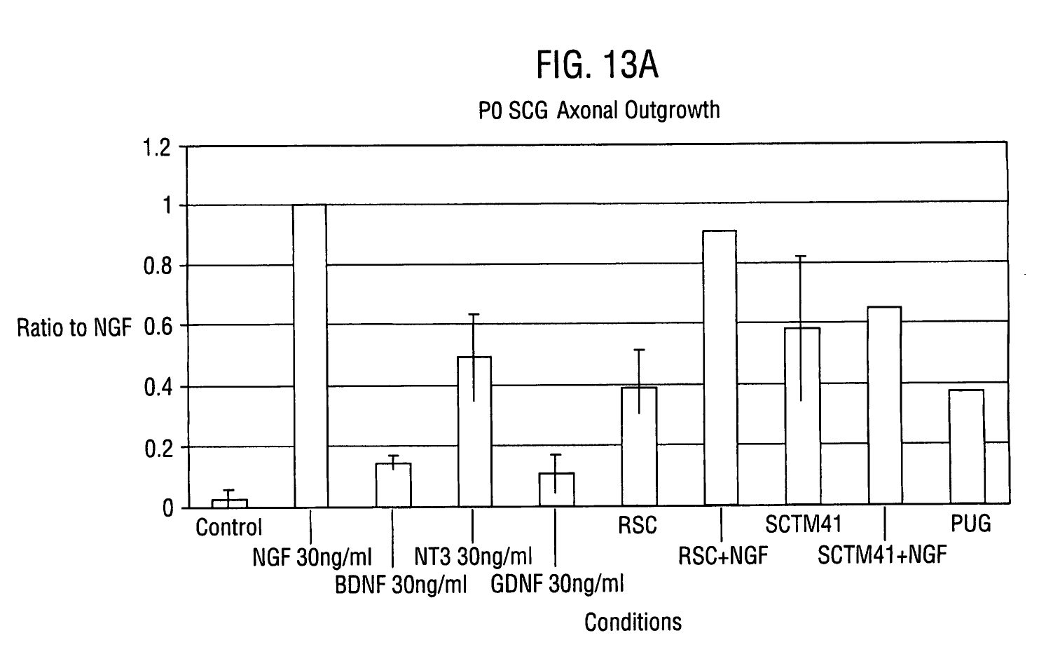

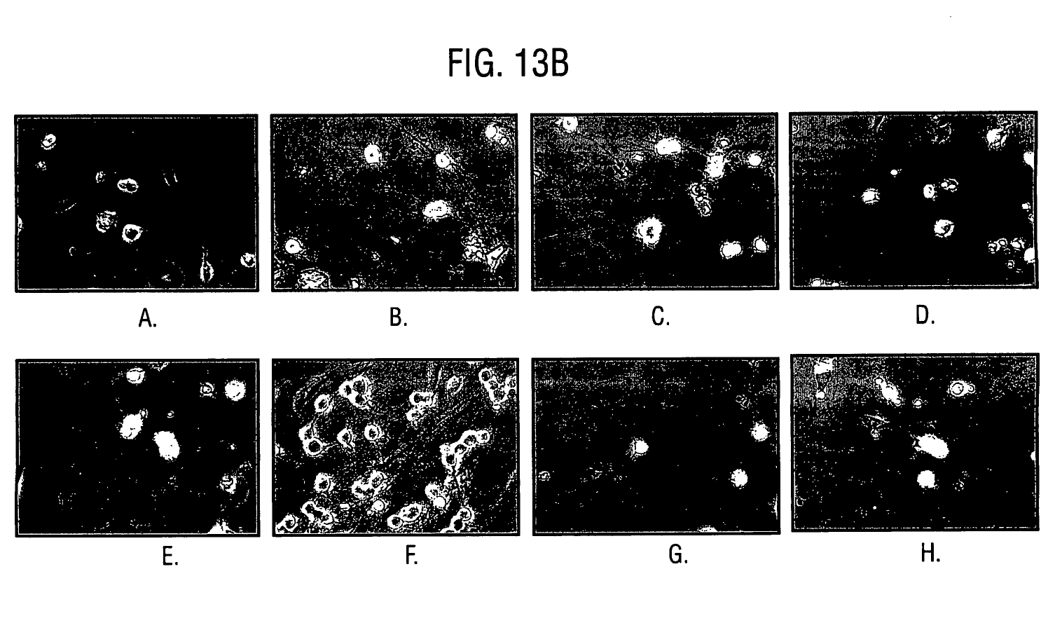

By postnatal day zero, mammalian sympathetic neurons have switched from trophic dependence on NT3 to dependence upon NGF. Consistent with this observation, axogenesis in cultures of sympathetic neurons derived from the P0 superior cervical ganglia and stellate ganglia when cultured in the presence of 50 ng/ml NT3 demonstrated only 49+14% of the axon outgrowth seen when cultured in the same concentration of NGF (see FIG. 13A). Likewise, a switch in Schwann cell or Schwann cell line conditioned media encouraging the greatest growth was observed from an embryonic preference of RSC conditioned media to SCTM41 conditioned media. Axonal outgrowth in the former was 39+12% that observed in NGF, while the latter was associated with outgrowth 58+24% that of NGF. The addition of NGF to RSC increased the axonal outgrowth to 91% that of NGF. The axonal outgrowth associated with culture in PUG was similar to that in RSC, exhibiting 37% of the growth associated with NGF. Cultures of neurons in the presence of neurotrophins BDNF and GDNF demonstrated growth greater than that of control (2.6+3.3% NGF) but markedly less than either NGF or NT3. Images of cells under different conditions of culture media, viewed by phase contrast microscopy, are presented in FIG. 13B.

To correlate the activity of conditioned media with that observed in the presence of neurotrophins, P0 sympathethic neurons were also cultured in conditioned media to which anti-trk receptor antibodies had been added. By using Trk-receptor bodies, neurotrophin action can be selectively eliminated (Eg. Trk-A receptor body destroys the effect of SCTM41 (NGF-dependent action) but has little effect on SCM).

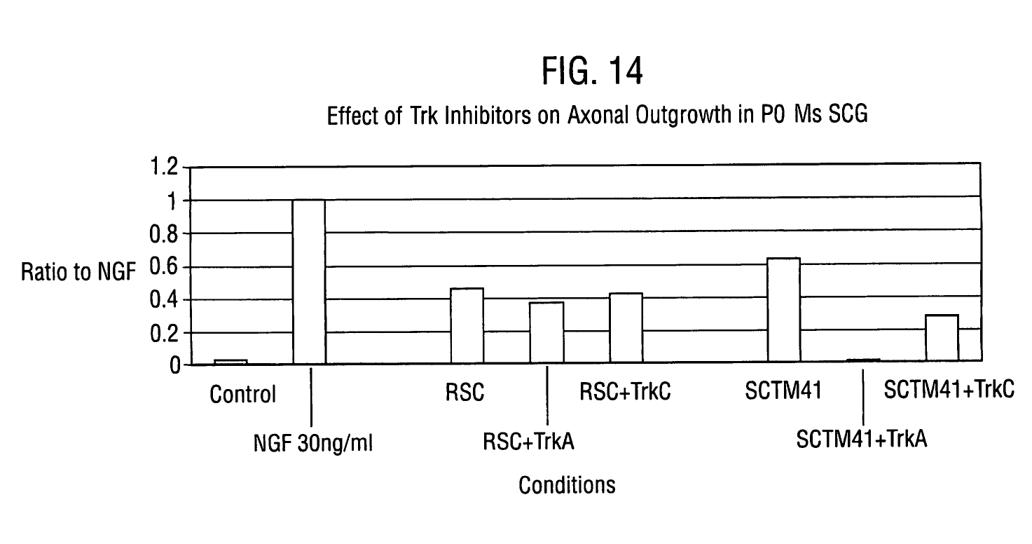

As observed in axonal outgrowth in cultures of dorsal root ganglia neurons, addition of neither the anti-trkA nor the anti-trkC receptor antibody to primary rat Schwann cell conditioned media (RSC) appeared to reduce neuronal outgrowth (see FIG. 14). In sympathetic cultures to which RSC+anti-trkA had been added, axonal outgrowth was 37% that observed with NGF. In sympathetic cultures to which RSC and anti-trkC receptor antibody had been added, the outgrowth was 42% that observed in culture with NGF. Additionally consistent with previous observations, culture of sympathetic neurons in SCTM41 and anti-trkA receptor antibody almost completely eliminated axonal outgrowth (1% of NGF), while the combination of SCTM41 and anti-trkC receptor antibody diminished, but did not eliminate, axonal outgrowth (to 28% of that observed with NGF). Use of all three Trk receptor bodies shows the effects of SCM are not due to Trk-mediated neurotrophins.

These latter experiments clearly show that the Schwann cell medium effects are not neurotrophin dependent.

### Example 5

Further Analysis of the Effect of Osteonectin Protein on Neuronal Survival and Regeneration

Preliminary data generated by further studies of retinal ganglion cell neurons, in this case using P8 rat RGCs, suggest that osteonectin increases survival and neuritogenesis of P8 rat RGCs when compared to control conditions (data not shown) and that there are both direct and indirect effects mediated synergistically with neurotrophins.

To explore the synergistic effects of osteonectin further, and to look at a peripheral neuron type in which Schwann cell mediated effects may be physiologically more relevant, further studies were undertaken using the well-characterised model of sympathetic neurons. Sympathetic neurons (SGCs), as used in Example 4, can be isolated from postnatal rodent superior cervical ganglia, purified to a high level and cultured in standardised conditions. The dependence of sympathetic neurons on one neurotrophic factor, Nerve Growth factor (NGF), makes them amenable as an experimental model.

SGCs were grown under control conditions with 10 ng/ml of NGF, which results in optimal survival and regeneration, as revealed by neurite outgrowth. The effects of osteonectin were quantified using an image analysis system, which enables the area of neuronal cell soma to be subtracted from the total area of neurite as revealed by anti-neurofilament antibody staining. The following materials and methods were used in these investigations:

Postnatal day 1 mice (SV129 bred in house) were killed by terminal anaesthesia (sodium pentobarbitone /gm). The head was removed and the superior cervical ganglion exposed on both sides. Ganglia were removed and kept on ice in Hanks' balanced salt solution. Ganglia were dissected free of surrounding tissues and placed in an enzyme cocktail of 0.05% collagenase, 0.005% DNAse I/I for 40 minutes at 37° C. to remove the collagen sheath.

Desheathed ganglia were digested in trypsin (sigma) for 18 minutes at 37° C. Ganglia were washed in DMEM with 10FCS then gently dissected using a fire polished pipette. Sympathetic neurons were purified by plating onto collagen dishes, to which fibroblasts adhere preferentially. After 20 minutes plating non adherent cells were placed on a second collagen plate to complete purification. The purified neurons were collected, spun at 2K for 3 minutes and re-suspended in DMEM-f12 with FCS.

Neurons were plated at 500, 2000 and 5000 neurons per poly-l-lysine/laminin coated 13 mm coverslip in a 4 well culture plate. All cultures were grown for 48 hours before fixation in 4% paraformaldehyde with 20% sucrose. Immunostaining was carried out using an anti-neurofilament antibody (3A10) flowed by a FITC labelled goat anti mouse IgG second anti-body and DAPI counterstaining of the cell nuclei. Images were digitally photographed at ×10 magnification and analysed using Kontron KS400 image analysis software. Three macros were written to count (i) total fluorescent tissue (all neural cells and neurites) (ii) cells bodies alone, which allowed a neurite measure to be made by subtraction (iii) total neuronal number based on DAPI labelling, which allowed neurite per cell to be calculated.

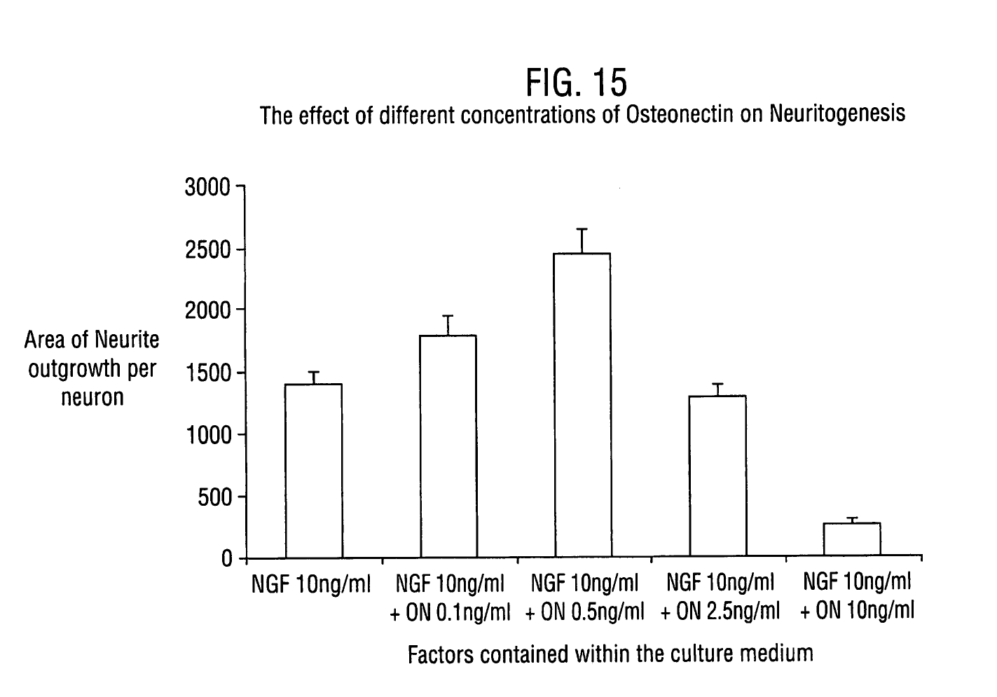

In order to optimise the level of osteonectin in SCG cultures, a dose response analysis was undertaken. SGCs were grown under control conditions with 10 ng/ml NGF and then osteonectin added in increasing doses (results shown in FIG. 15). Optimal survival and neurite outgrowth were achieved with 0.5 ng/ml of osteonectin. Further increases in the level of osteonectin produced short stunted neurite outgrowth and inhibited the regeneration response.

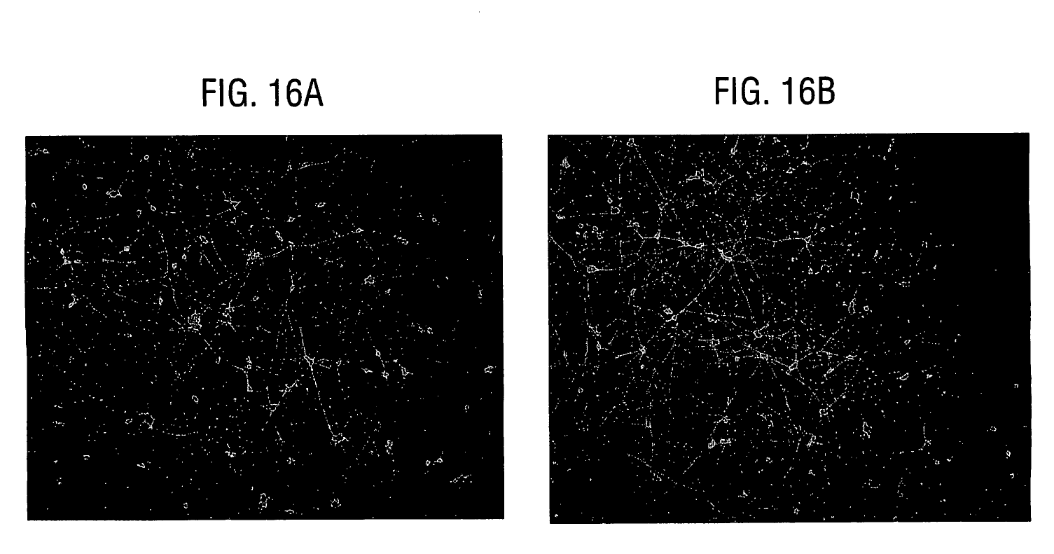

FIG. 16 shows the results of an experiment in which P4 mouse sympathetic neurons were cultured in medium with 10 ng/ml NGF at 5000/per well, in absence A) or presence B) of 0.5 ng/ml osteonectin. In B there appears to be an increased number of neurites stained with anti2H3 antibody, which binds to 165 KD neurofilaments.

Therefore, fluorescent imaging of the SGC cultures reveals enhanced neurite outgrowth when osteonectin is present in the culture medium, suggesting osteonectin acts synergistically with NGF.

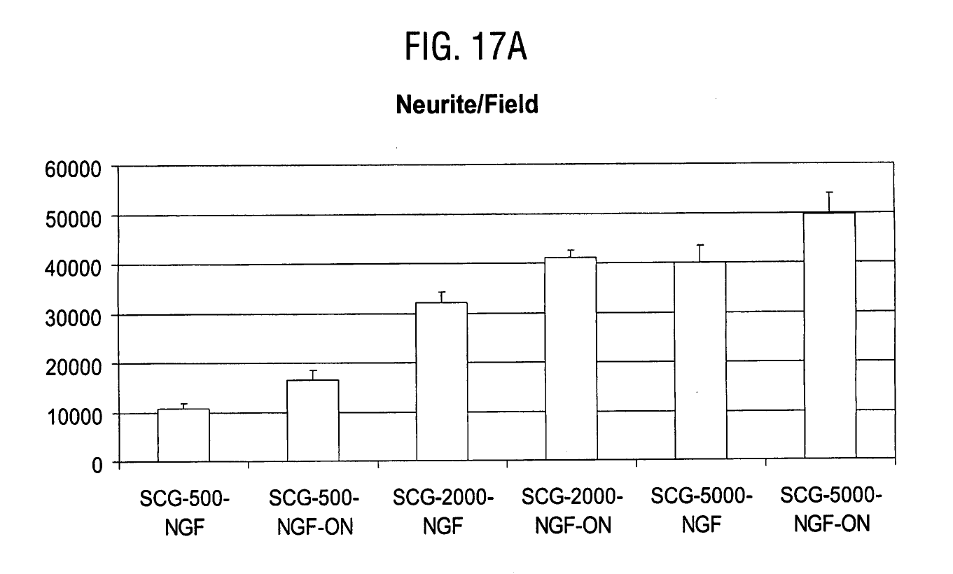

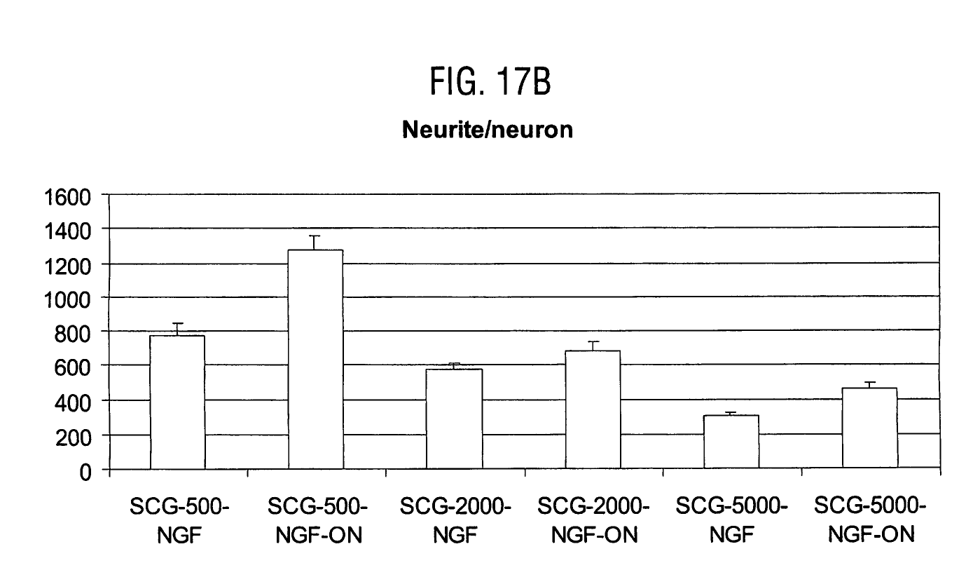

Experiments were also performed using cells at low and high density to allow a determination of whether there is an autocrine cell-cell interaction which is also acting on the SCGs. FIG. 17A provides a graph showing the quantification of the effects of the optimal dose of 0.5 ng/ml of osteonectin, in cultures of sympathetic neurons at varying densities, grown in the presence of 10 ng/ml NGF. Osteonectin is found to increase neurite outgrowth at all cell densities examined, again showing that it is acting synergistically with NGF.

Notably, as cell number increases in the culture, the effects of both NGF and NGF+osteonectin (ON) are potentiated. This suggests that other factors released by the SCG neurones themselves are also acting positively on the cells. This could be either a direct interaction with osteonectin or NGF or by an unrelated second cell survival neuritogenesis pathway.

In addition, the use of an image analysis system allows total neuronal number to be assayed and then a calculation of neurite per soma to be made. Accordingly, FIG. 17B shows the effects of osteonectin on the number of neurites per neuron. The higher cell densities show a reduction in neurite number per neuron probably due to contact inhibition. Osteonectin increases the number of neurites in all cultures examined.

Combined with the data presented in the previous Examples, these results show that osteonectin acts on both peripheral and central neurons and has effects which involve a complex interaction with other growth factors. Further experiments will now be undertaken to examine the downstream pathways acting on these cells, which will help identify the nature of these effects.

1. A method for stimulating the regeneration of a neuronal cell, said method comprising exposing the cell to the osteonectin protein or a functional equivalent thereof in an amount that is effective to stimulate the regeneration of the cell.

2. A method according to claim 1, wherein the neuronal cell is a CNS neuronal cell.

3. A method according to claim 1, wherein the cell is exposed to the osteonectin protein or functional equivalent thereof in vitro.

4. A method according to claim 1, where the cell is exposed to the osteonectin protein or functional equivalent thereof in vivo in a patient

5. A method according to claim 4, wherein the neuronal cell is exposed to the osteonectin protein or functional equivalent thereof through the introduction into a patient of a nucleic acid molecule that expresses the osteonectin protein or functional equivalent thereof.

6. A method according to claim 5, wherein the neuronal cell is exposed to the osteonectin protein or a fragment or variant thereof through the introduction into a patient of a cell that contains the nucleic acid molecule which expresses the osteonectin protein or a functional equivalent thereof in a therapeutically-effective amount.

7. A method according to claim 4, wherein said neuronal cell is exposed to a pharmaceutical composition comprising the osteonectin protein or functional equivalent thereof in an amount appropriate to stimulate the regeneration of the cell.

8. A method according to claim 7, wherein said pharmaceutical composition comprises a cell which expresses the osteonectin protein or a functional equivalent thereof.

9. A method according to claim 6, wherein said cell is derived from the patient being treated.

10. A method according to claim 1, wherein said neuronal cell is exposed to the osteonectin protein or functional equivalent thereof in the presence of one or more additional neurotrophic factors.

11. A method according to claim 10, wherein said one or more neurotrophic factors is selected from the group consisting of nerve growth factor, glial derived growth factor, brain derived growth factor, ciliary neurotrophic factor, neurotropin-3, vascular endothelial growth factor (VEGF), fibroblast growth factor (FGF) and platelet-derived growth factor (PDGF).

12. A method according to claim 1, wherein said neuronal cell is exposed to the osteonectin protein or functional equivalent thereof impregnated into an artificial bridging substrate which is placed at or near the site in the body at which neuronal regeneration is required.

13. A method according to claim 12, wherein said artificial bridging substrate is Elvax®.

14. A method according to claim 1, wherein said stimulation of the regeneration of a neuronal cell is in the context of the treatment of a disease selected from the group consisting of peripheral neuron damage, such as by physical injury or disease state such as diabetes, in the case of injury or a disease state of the CNS, including physical damage to the spinal cord, brain trauma, stroke, retinal and optic nerve lesions, neurodegenerative diseases such as Alzheimer's disease and Parkinson's disease, neuromuscular diseases, autoimmune diseases of the nervous system, tumours of the central nervous system, damage to motor neurons such as occurs in amyotrophic lateral sclerosis, retinitis pigmentosa or age-related macular degeneration

15. Use of the osteonectin protein or a functional equivalent thereof in the manufacture of a medicament for the stimulation of neuronal cell regeneration, preferably, for the stimulation of CNS neuronal cell regeneration.

16. Use according to claim 15, wherein said osteonectin protein or a functional equivalent thereof is used in the manufacture of a medicament for the treatment of peripheral nerve damage, such as by physical injury or disease state such as diabetes, in the case of injury or a disease state of the CNS, including physical damage to the spinal cord, brain trauma, stroke, retinal and optic nerve lesions, neurodegenerative diseases such as Alzheimer's disease and Parkinson's disease, neuromuscular diseases, autoimmune diseases of the nervous system, tumours of the central nervous system, damage to motor neurons such as occurs in amyotrophic lateral sclerosis, and retinitis pigmentosa and age-related macular degeneration

17. A method of screening for a receptor protein that mediates the stimulation of neuronal cell regeneration, said method comprising exposing a cell to osteonectin, cross-linking the osteonectin protein to a receptor on the cell, and analyzing the complex to identify the receptor.

18. A method of screening for a receptor protein that mediates the stimulation of neuronal cell regeneration, said method comprising contacting a candidate cell with the osteonectin protein or a functional equivalent thereof and assessing the ability of the cell to effect stimulation of neuronal cell regeneration

19. A method according to claim 18, wherein a library of candidate cells is exposed to the osteonectin protein or a functional equivalent thereof.

20. A method according to claim 19, wherein said library of candidate cells has been transformed with a library of nucleic acid molecules, each nucleic acid molecule encoding a candidate receptor for osteonectin.


SEQUENCE LISTING (1 sequence)

SEQ ID NO: 1 - protein, 15 aa, Homo sapiens
  Amino acid sequence derived from N-terminal Edman sequencing
APQTEAAEEMVAEET